(19) United States
(12) Patent Application Publication
(10) Pub. No.: US 2007/0203091 A1
(43) Pub. Date: Aug. 30, 2007

(54) Methods and therapeutic compositions for improving liver, blood flow and skeletal muscle functions in advanced diseases and aging

(76) Inventor: Eliezer Rapaport, Belmont, MA (US)

Correspondence Address: CONNOLLY BOVE LODGE & HUTZ LLP, 1875 EYE STREET, N.W., SUITE 1100, WASHINGTON, DC, 20036, US

(21) Appl. No.: 11/362,855

(22) Filed: Feb. 28, 2006

(51) Int. Cl. A61K 31/7076

(52) U.S. Cl. 514/47

(57) ABSTRACT

Administration of adenosine 5′-triphosphate (ATP) and/or other adenine nucleotides such as adenosine 5′-monophosphate (AMP) and/or adenosine 5′-diphosphate (ADP) and/or adenosine provides significant benefits to liver, blood flow and skeletal muscle functions in humans suffering from advanced diseases or in aging individuals. In a preferred mode, 8 hours of continuous intravenous infusions of 10-100 microgram/kg·minute of ATP in an out-patient setting, is shown to stabilize primary independent negative prognostic markers of survival and quality of life in terminal aging cancer patients suffering from serious clinical deterioration due to the advanced disease. During aging or advanced diseases that afflict the aged, systemic organ failure is initiated. ATP treatment provides benefits by stabilizing independent negative prognostic markers of survival and preventing the serious clinical deterioration that normally follows.

### TECHNICAL FIELD

The present disclosure relates to the treatment of patients suffering from advanced diseases originating with organ failure or aging humans with adenine nucleotides and especially adenosine 5′-triphosphate (ATP). The resulting improvements in liver function, stimulation of blood flow and skeletal muscle strength have positive effects on survival and quality of life of these individuals.

### BACKGROUND ART

Adenosine 5′-triphosphate has been established as the major cellular energy source, an intermediate in a great variety of intracellular synthetic reactions, a phosphate donor and an allosteric regulator of the activities of cellular proteins. ATP has also been shown to act extracellularly, as a major in vivo regulator of metabolic, vascular and muscle functions in humans (1, 2). Its extracellular activities are mediated through interactions with a family of ATP receptors (P2 receptors) that are present on the membrane of virtually every cell. The in vivo catabolic (degradation) product of ATP, adenosine, interacts with its own family of receptors (A receptors) and possesses major regulatory roles as well.

Administration of ATP in experimental animals or humans, results in the expansions of liver, blood (red blood cells) and blood plasma (extracellular) pools (steady state levels) of ATP (3, 4). The administration of exogenous ATP, or any other adenine nucleotide, in a suitable formulation, results in a rapid degradation of the adenine nucleotide to adenosine and inorganic phosphate inside the vascular bed. Both adenosine and inorganic phosphate are then incorporated into the liver ATP pools, yielding expansions of these pools. Detailed studies in animals along with human clinical trials have shown that the turnover of the expanded liver ATP pools, supply increased adenosine precursor, in the hepatic sinusoids, for enhanced synthesis of ATP in red blood cells (3-5). Mature red blood cells utilize only a salvage precursor (adenosine) for the synthesis of ATP by a glycolytic pathway only. The red blood cells containing elevated ATP pools, slowly release ATP into the blood plasma (extracellular) compartment by a non-hemolytic mechanism. It is the increased levels of liver, red blood cell and blood plasma ATP, which are of primary importance in improving a great variety of physical functions. The half-life of expanded ATP pools in red blood cells is about 6 hours (3,4,6) and the release of ATP from red blood cells yields increase levels of ATP and its degradation product, adenosine, extracellularly in the blood plasma. This process is regulated by physiological mechanisms that produce these agents inside the vascular bed at sites and times where and when they are needed (7-9), mostly in responding to the metabolic demands of contracting skeletal muscle. ATP and adenosine are known powerful vasodilators inside the vascular bed, acting through interactions with P2Y and A2 receptors present on vascular endothelial cells. This mechanism that produces an immediate increase in blood flow needed to meet the metabolic demands of hypoxic (oxygen poor) tissues is strictly dependent on ATP release from red blood cells (7-9).

During aging (e.g. 65-75 years old), initial levels of red blood cell ATP pools drop to about half of what they are in young individuals (10). Older humans (mean age of 68.8 years) retain only 50% of muscle mitochondrial ATP synthesis as compared with adults (mean age of 38.8 years) (11). Purine (ATP and adenosine) losses, adversely affecting organ and skeletal muscle functions, were also reported in diseases and other stressful conditions (1). The reduced blood and skeletal muscle pools of ATP in the aged, lead to a variety of adverse conditions, which are primarily the result of decreased blood flow.

Animal studies showed that low levels of ATP administered directly into the duodenum, the proximal part of the small intestine, yielded significant positive cardiovascular and pulmonary responses (12). These included reductions in pulmonary vascular resistance, reductions in peripheral vascular resistance followed by increases in blood flow. No effects on arterial blood pressure or heart rate were observed. An increase in left ventricular work index, which is an indication of improved cardiac output was found. Cardiac output is a value that expresses the efficiency of the heart in circulating the blood throughout the vascular bed and is expressed in units of L/min/sq m. In addition, an increase in arterial oxygen pressure (PaO₂) was observed after the administration of ATP. Intraluminal ATP, at physiological concentrations, was shown to produce not only local vasodilation, but also vasodilation at sites upstream from the site of its application. Adenosine on the other hand, induced only local vasodilation. Low physiological levels of blood plasma ATP (about 1 microM), induced 8% increase in vascular diameter, corresponding to a minimum of 17% increase in blood flow (13). Vasodilation induced by physiological levels of ATP is mediated primarily by nitric oxide (NO), which is synthesized by the enzyme NO synthetase in vascular endothelial cells in response to the interaction of ATP with P2Y receptors. The NO then diffuses into and acts in neighboring perivascular smooth muscle cells, which control vascular tone and produce relaxation and vasodilation of the blood vessel in response to NO. At higher levels of ATP, corresponding to ATP released from red blood cells containing expanded ATP pools, other mechanisms of vasodilation operate besides NO synthesis. These mechanisms include induction of vasodilatory prostaglandins synthesis, mostly prostacyclin (PGI₂) as well as non-NO, non-prostacyclin induced vasodilation that is mediated by the direct interactions of ATP and adenosine with their corresponding receptors (13).

The direct correlation between aging and the decline mostly in skeletal muscle mitochondrial ATP synthesis (11, 14) as well as the significant decreases in blood ATP parameters upon aging in humans (1,10) and experimental animals (15,16) have been established. Recently however, decreases in ATP levels caused by intentionally introduced mutation into mitochondrial DNA in animals (17) and declines in skeletal muscle mitochondrial function in humans (18) were demonstrated to be a direct cause of aging. Thus, a direct relationship between significant declines in skeletal muscle and blood levels of ATP and the aging process has now been established (17, 18).

The desire to slow the aging process by improving skeletal muscle strength and function has attracted a considerable degree of interest. Hormone treatments of elderly men with human growth hormone (GH) and testosterone and hormone treatment of elderly women with GH and hormone replacement therapy (HRT), was the subject of a recent large clinical trial (19). The results confirmed the apparent positive effects of growth hormone and sex steroid combinations on body composition, namely, increasing lean body mass and decreasing fat mass (19). However, the results clearly demonstrated that lean body mass did not translate into improved skeletal muscle function and as importantly, the risk of adverse effects associated with the use of these hormonal regimens was substantial (20).

U.S. Pat. No. 5,049,372 to Rapaport discloses a process for increasing blood and plasma levels of ATP by administration of adenine nucleotides or adenosine and utilization of the elevated ATP pools for inhibition of tumor growth and host weight loss in cancer. U.S. Pat. No. 5,227,371 to Rapaport discloses a method and process for increasing total liver, blood and blood plasma ATP pools by administration of adenine nucleotides or adenosine.

U.S. patent application Ser. No. 08/131,948, entitled “Methods of Treatment of Human Immunodeficiency Virus (HIV) Disease and Acquired Immunodeficiency Syndrome (AIDS) in a Human Host by Administration of Adenine Nucleotides”, Filed Oct. 8, 1993 discloses the utilization of expansions of liver, blood (red blood cell) and blood plasma ATP pools for the improvements of liver, blood flow and skeletal muscle functions. The improvement in hepatic function after administration of ATP was demonstrated to be linked to the expansions of liver ATP pools. The positive effects on skeletal muscle functions and body composition after administration of ATP were shown to be the result of expansions of liver, red blood cell and blood plasma ATP pools, which in turn resulted in significant improvements in blood flow to peripheral sites. The direct relationship of blood flow to skeletal muscle function was later confirmed in the art (7-9). The disclosures of benefits to hepatic, blood flow and skeletal muscle functions were later confirmed by administration of ATP to cachectic, advanced refractory cancer patients. In this regard intravenous administration of ATP to this patient population was shown to contribute to global beneficial effects (21-23) including survival advantages (24) for patients receiving ATP versus a control group receiving best supportive care.

Recently, it was shown that in Chronic Obstructive Pulmonary Disease (COPD), traditional measures such as spirometry, correlated poorly with the major clinical end-points of survival and quality of life (25). It was concluded that at the advanced stage, COPD along with other advanced pulmonary diseases are systemic diseases where the systemic effects due to multiple organ failure substantially contribute to morbidity and mortality (25). Improvements in hepatic functions and skeletal muscle strength are expected to produce survival and quality of life benefits. Other examples where systemic aspects due to organ failure, rather than localized effects of the particular advanced disease, significantly contribute to morbidity and mortality are acute lung injury (ALI) and acute respiratory distress syndrome (ARDS) (26). Muscle wasting is encountered in a variety of terminal conditions in addition to advanced refractory cancer and severe pulmonary diseases. These include rheumatoid arthritis, diabetes, heart failure, severe injury, kidney disease and sepsis (27). Not surprisingly, muscle wasting or cachexia is a major independent negative prognostic factor in all of these diseases.

### SUMMARY

The present disclosure establishes that independent negative prognostic factors of quality of life and survival in the terminal stages of a variety of advanced diseases that afflict mostly the elderly and aging itself, can benefit from treatment with adenine nucleotides such as ATP and/or ADP and/or AMP and/or adenosine. The reason for the common features of advanced diseases and aging is the systemic nature of the serious clinical deterioration, which originates in organ failure. My vast experience in the utilization of adenine nucleotides and continuous intravenous infusions of ATP in particular, along with a number of unrelated observations and properties of ATP and adenosine have enabled me to conclude that this type of systemic organ failure can benefit by administration of adenine nucleotides to individuals in need of, as is disclosed in this application. In order to demonstrate the invention in a non-limiting fashion, I selected a group of older patients suffering from serious clinical deterioration of advanced, refractory (patients who have failed surgery, chemo-and/or radiation therapy) terminal cancers. The primary independent negative prognostic factors of survival that significantly benefited from ATP administration were serum albumin and serum bilirubin levels, serum lactate dehydrogenase (LDH) levels, blood levels of tumor necrosis factor-alpha (TNF-alpha), skeletal muscle strength and Karnofsky performance status, all of which are also known to be significant quality of life determinants. All blood parameters of ATP were elevated after administration of exogenous ATP.

The present application discloses methods for the improvement of liver function, for the stimulation of blood flow and for the increase in skeletal muscle strength in aging humans and in patients suffering from advanced systemic diseases.

In particular, the present disclosure is concerned with methods and processes for the improvement of quality of life in aging individuals and in patients suffering from advanced, end-stage diseases with systemic multiple organ failure. The administration of active agents at home, in an out-patient setting or in a clinic, results in increases in liver and total blood (red blood cell) ATP pools. The rate of release of ATP from red blood cells into the blood plasma (extracellular) compartment is enhanced, resulting in elevated blood plasma ATP pools. These improvements in physiological ATP pools are directly responsible for the claimed benefits to physiological functions of humans benefiting from the claimed treatment.

It has been established recently that skeletal muscle ATP pools and total adenine nucleotide (TAN) pools are reduced by about 20% in healthy individuals of a mean age of 65 years, exercising for a short period of time (five minutes) at 80% work peak. Chronic obstructive pulmonary disease (COPD) patients of the same mean age are capable of exercising at a much lower work load as compared to the healthy controls. COPD patients also lose about 20-25% of skeletal muscle ATP pools and total adenine nucleotide pools during such short period exercise (28). However, the initial skeletal muscle pools of ATP and total adenine nucleotides at rest in COPD patients are significantly and dramatically lower by about 25% than the same pools in healthy controls of the same mean age (65 years) (28). The inability of COPD patients to recover their ATP and total adenine nucleotide pools after muscle contraction is responsible for their significantly lower skeletal muscle pools of these metabolites, resulting in the COPD patients capable of performing only about 40% of the work load of healthy controls (28).

The present disclosure teaches that the three physiological functions, liver functions, blood flow and skeletal muscle functions can benefit in aged individuals or in patients suffering from advanced, terminal diseases by administration of ATP and/or other adenine nucleotides and/or adenosine. By aging individuals, is meant those at least 60 years old.

### BEST AND VARIOUS MODES

It has been found pursuant to the present disclosure that aged individuals suffering from advanced, refractory, terminal stage cancers benefit by being administered a member selected from the group consisting of: (a) adenosine; and (b) an adenine nucleotide wherein said adenine nucleotide is ATP and/or ADP and/or AMP. This advanced, terminal, systemic disease is utilized in a non-limiting fashion to demonstrate the broader nature of claimed treatment.

Preparations containing the above ingredients can be employed in a variety of conventional pharmaceutical preparations. These preparations can contain organic or inorganic material suitable for internal administration. The high solubility of AMP and/or ADP and/or ATP salts and/or adenosine with or without inorganic phosphate salts in isotonic aqueous solutions of sodium chloride enable administration of these agents in the form of injection or infusion of single or multiple doses. The injection or infusion can be intraperitoneal, intravenous, or intra-arterial. AMP and/or ADP and/or ATP and/or adenosine are also suitable for oral, enteral, or topical application when employed with conventional organic or inorganic carrier substances.

The effective doses are in the range of about 0.01-50 mg/kg of body weight per 24 hours for oral, sublingual or topical administration, and 0.01-50 mg/kg of body weight per 24 hours for injections. Continuous intravenous, intraperitoneal, or intraarterial infusions of AMP and/or ADP and/or ATP and/or adenosine in a suitable salt form are preferably administered at a rate of about 0.001-0.15 mg/kg of body weight per minute. In a preferred mode, 8 hours of continuous intravenous infusions of 10-100 microgram/kg·minute of ATP in an out-patient setting, is shown to stabilize primary independent negative prognostic markers of survival and quality of life in terminal aging cancer patients suffering from serious clinical deterioration due to the advanced disease. The delivery of active agents by continuous intravenous infusion can be performed in an out-patient setting including and sometimes preferred a home infusion setting with or without medical supervision. The delivery of these agents can be performed using a variety of drug delivery systems including, but not limited to, pumps or liposomes. In addition, pharmaceutically acceptable salts, or metal complexes, or chelates, or liposomes, or radio-nuclides of the above compounds can be used.

An example of a clinical procedure in the treatment of individuals in need thereof is as follows. After determination of baseline, pre-treatment vital signs, hemodynamic variables and blood chemistry, an ATP dose escalation procedure is initiated. ATP is provided as a sterile solution in single use vials. Each vial contains 2 grams of disodium ATP in 20 ml of Water for Injection. The concentration of ATP is 100 mg/ml. Storage of the clinical solution is at controlled refrigerated temperature (2° C.-6° C.). Preparation of the infusion solution requires that the volume of one vial of ATP be aseptically removed using a syringe and added to a 250 ml bag of 0.5 normal saline (which has been volume corrected by removal of 20 ml of saline). The concentration of the final sterile solution for the infusion is 8 mg/ml. The stability of the final ATP solutions at room temperature is at least 96 hours. The preparation of ATP can be in a vial in a lyophilized form with suitable excipients and the administration of ATP can be performed by the use of a home infusion pump at the patient's home with or without medical supervision.

Pharmacology of ATP.

Cardiovascular Effects.

A number of studies that have described the effects of continuous intravenous infusions of ATP on the cardiovascular system in anesthetized animals are summarized in Table 1.

TABLE 1
Effects of Intravenous Infusions of ATP in Experimental Animals

| | ATP Infusion Rate | |
|---|---|---|
| Parameters | ≦100 μg/kg/min | 500-1000 μg/kg/min |
| Heart Rate | No Change | Increase |
| Systemic Arterial Pressure | No Change | Decrease |
| Pulmonary Arterial Pressure | No Change | Decrease |
| Cardiac Output | Small Increase | Increase |
| Systemic Vascular Resistance | Small Decrease | Decrease |
| Pulmonary Vascular Resistance | Decrease | Decrease |

At relatively low rates of infusions, below 100 micrograms per kg of body weight per minute, intravenous infusions of ATP produced changes primarily in pulmonary hemodynamics, with little, if any, changes in the systemic circulation. These infusions decrease pulmonary vascular resistance, but do not affect systemic arterial pressure or heart rate although there is a small decrease in systemic vascular resistance and a small increase in cardiac output. It is the powerful vasodilation produced by ATP and adenosine inside the vascular bed, leading to the stimulation of blood flow without affecting arterial blood pressure or heart rate that is the basis for the anti-aging efficacy of exogenously-administered ATP.

Physical, Chemical, and Pharmaceutical Properties (Drug Product)

In a typical formulation, ATP is provided as sterile liquid in 20 ml vials. Each vial contains 2.0 grams of ATP in 20 ml of water for injection USP (100 mg ATP/ml), pH adjusted with sodium hydroxide to 6.8-7.1. The product is desirably stored at controlled refrigerated temperature (2° C.-6° C.).
Structure: 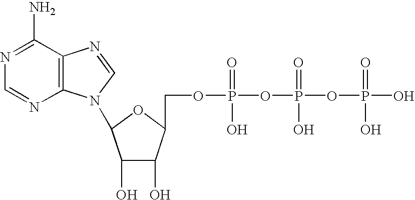
Drug Distribution, Metabolism, and Elimination.
Distribution.

ATP is widely distributed, being found in every cell.

Metabolism.

It is well known that ATP is metabolized in man and the Dalmatian dog via the following series of metabolites: (1) adenosine 5′ diphosphate (ADP), (2) adenosine monophosphate (AMP), (3) adenosine, (4) inosine, (5) hypoxanthine, (6) xanthine, and (7) uric acid. In other mammals, uric acid is oxidized to allantoin. Many of these intermediates are recycled back into selected biochemical pathways in most organs but the kidney, each to a variable extent dependent on the species, excretes uric acid and allantoin.

Elimination.

As mentioned above, ATP is metabolized in several steps to uric acid, and in some species to allantoin. These metabolites are then excreted by the kidney in a species-dependent manner.

Safety and Efficacy.

Although rare, the following serious and potentially life-threatening complications have been associated with intravenous infusions of adenosine (a known metabolite of ATP) when adenosine infusion was at a rate higher than the planned rate of infusion of ATP: severe bronchospasm (0.03%), nonfatal myocardial infarction (0.02%), severe hypotension (0.45%), and severe bradycardia (0.04%). Such complications should be managed as clinically indicated and with recording of the event on the case report form.

Applicant is the sole inventor of the invention disclosed and taught in the present application. The inventor is also the sole and exclusive owner of the data presented in the present application.

The data presented here have not been published yet and include parts of a copy of an annual report to the FDA of Oct. 15, 2001 and parts of copies of a final analysis concluded on Sep. 21, 2005. The results are of a clinical study entitled “A Phase I Study of the Safety and Pharmacokinetics of Adenosine 5′-Triphosphate (ATP) When Administered by Intravenous Infusion on a Multiple Weekly Dose Schedule to Patients with Advanced Malignancies (Solid Tumors)”. The annual report to the FDA (pages 15-1 to 15-11) outlines detailed interim analysis of the first 9 patients enrolled in the trial. The final analysis includes data of 15 patients enrolled in the trial.

The patients enrolled in this trial were cancer patients suffering from advanced, refractory, terminal disease, who failed surgery and chemo-and/or radiation therapy and had short life expectancy. Five of the fifteen patients had to be withdrawn during the trial due to serious clinical deterioration (SCD). Fourteen out of the fifteen patients had secondary tumors, including several patients with brain metastasis. The clinical status of the patients entering the trial is described on pages 15-20 and 15-21, Table 6 and Table 16.2.4.1 respectively. Most of the patients were clinically unstable and some experienced a drop in Karnofsky Performance Status between screening and the beginning of the trial protocol (page 15-11).

The patients participating in this clinical trial comprised an example of the individuals in need of claimed treatment. Namely, they tended to be older, with a mean age of 60.6 years and suffered from an advanced, refractory, terminal disease (pages 15-20, 15-21, Tables 6 and 16.2.4.1).

The protocol of the clinical trial is described on pages 15-14 to 15-18.

Administration of ATP continuously for eight hours in accordance with the protocol, resulted in increases in total blood (red blood cell) ATP levels (pages 15-22, 15-23, Table 14.2.11), increases in the initial ATP release rate from red blood cells into the blood plasma compartment (pages 15-24, 15-25, Table 14.2.11) and increases in blood plasma (extracellular) ATP levels (pages 15-26, 15-27, Table 14.2.11). The observed increases in these three blood ATP parameters upon administration of ATP in humans, support claims reciting improvements in blood flow by claimed treatment in a human host in need thereof. It is now well established that elevated blood plasma ATP pools enhance blood flow, mostly to skeletal muscle, supporting contracting skeletal muscle metabolic demands for oxygen and nutrients as well as the enhanced removal of waste products such as lactic acid and ammonia. The relationship of blood plasma (extracellular) ATP levels to blood flow is discussed in enclosed references 7, 8 and 9.

Administration of ATP to human patients was shown in this clinical trial to stabilize and prevent a drop in serum albumin levels after 13 weeks, although levels of pre-albumin (which has a shorter half life than albumin) decreased and levels of C-Reactive Protein continued to increase (page 15-28, Table 14.2.6.1). Serum levels of lactate dehydrogenase (LDH) dropped at week 13 of treatment; whereas, serum levels of bilirubin remained stable at week 13 of treatment (page 15-29, Table 14.3.5.2). The data for albumin, LDH and Bilirubin support claims reciting improvement in liver function by claimed treatment of a human host in need thereof. Serum LDH levels are an established strong independent negative prognostic factor, inversely related to survival in a great variety of adverse clinical conditions. In advanced refractory cancers, high LDH and low Karnofsky's performance status are among the strongest established independent negative prognostic factors of survival and quality of life (29).

Administration of ATP was shown in the present trial to stabilize and improve skeletal muscle strength, beginning at week 8 and up to week 13 (page 15-30, Table 14.2.10 with a description of the measuring procedure on page 15-31). These data support claims reciting improvement in skeletal muscle function by claimed treatment of a human host in need thereof.

Administration of ATP was demonstrated in the present trial to stabilize the levels of blood tumor necrosis factor-alpha (TNF-alpha) and prevent increases in this cytokine levels at week 13 (page 15-32, Table 14.2.6.2). Interleukin-6 levels were not stabilized upon ATP treatment. TNF-alpha is an acknowledged independent negative prognostic factor of morbidity and mortality in the elderly (30).

Administration of ATP was shown in the present trial to stabilize Karnofsky Performance Status in this patient population (page 15-33, Table 14.2.7 and page 15-34 for definitions of Karnofsky scale). The present patient population was clinically deteriorating rapidly with four of the fifteen patients not meeting the protocol inclusion criteria and being granted exemptions (page 15-18). An amendment to the protocol had to be sought because Karnofsky Performance Status of some patients dropped between the screening date and the first pre-infusion date. Considering the clinical status of the present patient population at the start of the trial along with Karnofsky Performance Score being a surrogate end-point for Quality of Life in advanced, refractory, terminal cancers, these findings support claims reciting benefits to quality of life by claimed treatment in a human host in need thereof.

Karnofsky performance status, LDH and TNF-alpha are primary independent negative prognostic factors of morbidity and mortality in aging and/or in patients suffering from advanced terminal diseases. Serum albumin levels and skeletal muscle strength are strong factors positively affecting quality of life in these individuals. The present disclosure teaches a treatment that stabilizes or improves the levels of these parameters and thus benefits humans in need of claimed treatment. ATP administration elevated total blood (red blood cell) and blood plasma (extracellular) ATP pools along with increases in the rate of release of ATP from red blood cell into the blood plasma compartment. As a result and compared to baseline values, serum levels of albumin and bilirubin stabilized at normal levels, serum levels of lactate dehydrogenase (LDH) declined after a steady increase and blood levels of tumor necrosis factor-alpha (TNF-alpha) stabilized at baseline levels. In addition, the steady declines in skeletal muscle strength and Karnofsky performance status were halted by claimed treatment in this patient population.

During aging or advanced diseases that afflict the aged, systemic organ failure is initiated. ATP treatment provides benefits by stabilizing independent negative prognostic markers of survival and preventing the serious clinical deterioration that normally follows.

Typically individuals and/or patients are treated according to this disclosure for at least one condition selected from the group consisting of inflammatory bowel diseases, chronic heart diseases, chronic obstructive pulmonary disease, sepsis, acute lung injury, rheumatoid arthritis, osteoarthritis, advanced refractory cancer, severe trauma and injury, and more typically for at least one condition selected from the group consisting of inflammatory bowel diseases, chronic heart diseases, chronic obstructive pulmonary disease, sepsis, acute lung injury, rheumatoid arthritis, osteoarthritis, severe trauma and injury.

(a) (1) Individual Study Information:

Title of the Study: “A Phase I Study of the Safety and Pharmacokinetics of Adenosine 5′-Triphosphate (ATP) When Administered by Intravenous Infusion on a Multiple Weekly Dose Schedule to Patients with Advanced Malignacies (Solid Tumors)”.

Protocol Number: DMS #D0005, IND #60,517

The primary purpose of this study is to evaluate the safety, tolerability and pharmacokinetic properties of adenosine 5′-triphosphate (ATP) administered by continuous intravenous infusion to a maximum of 24 patients with histologically proven advanced treatment-resistant malignancies. The two secondary objectives of the study are to evaluate parameters that reflect quality of life and cancer cachexia in order to monitor any potential beneficial effects of ATP infusion in this patient population. Nine qualified patients have been enrolled in the study as of Sep. 1, 2001; seven of which received the first 3 cycles of the study drug and are therefore evaluable for the primary endpoint. Two of the seven evaluable patients had advanced prostate cancer with bone metastases and one each had advanced mesothelioma, metastatic breast cancer, metastatic melanoma, metastatic colon cancer and renal cell carcinoma.

DEMOGRAPHICS

| PATIENT | AGE | GENDER | RACE | CANCER TYPE | STATUS |
|---|---|---|---|---|---|
| 501 | 48 | Male | White, non-hispanic | Metastatic colon cancer | Withdrawn; progressive |
| | | | | | disease-N.E. |
| 502 | 54 | Male | White, non-hispanic | Metastatic mesothelioma | Completed study-Evaluable |
| 503 | 75 | Male | White, non-hispanic | Metastatic prostate | Completed study-Evaluable |
| | | | | cancer | |
| 504 | 77 | Female | White, non-hispanic | Metastatic breast cancer. | Completed study-Evaluable |
| 505 | 63 | Female | White, non-hispanic | Metastic melanoma to | Completed study-Evaluable |
| | | | | scalp | |
| 506 | 38 | Female | White, non-hispanic | Metastatic colon cancer | Completed 6 cycles- |
| | | | | | Evaluable |
| 507 | 69 | Male | White, non-hispanic | Metastatic prostate | Completed study-Evaluable |
| | | | | cancer | |
| 508 | 65 | Male | White, non-hispanic | Prostate cancer | Withdrawn, opted not to |
| | | | | | continue-N.E. |
| 509 | 48 | Female | White, non-hispanic | Renal cell carcinoma | Completed study-Evaluable |

*N.E. = not evaluable

Overall, patients tolerated the ATP infusion well, with seven of nine patients tolerating the maximum allowable dose of 100 μg/kg/min administered as continuous 8 hour intravenous infusion.

Patient enrollment has not been completed and the study remains open. However, due to an incident involving a personnel matter at the Dartmouth-Hitchcock Medical Center, the governing IRB has requested no new patients be consented to the study, as well as other studies involving this particular employee, until they have completed a review of the study operations involving this particular employee. The need for a review of the study operations is not directly related to the management of patients under the clinical protocol or related to patient safety concerns.

Patient-specific study drug administration information and patient narratives for the first nine patients enrolled are described in the following section.

(2) Study Patient Information

Summary as of Oct. 15, 2001

IND #60,517 (Dartmouth, DMS Protocol #D0005)

The enrollment numbers are as follows:

ENROLLED: 9 PATIENTS

| | |
|---|---|
| Active: | none |
| Discontinued: | 2 patients (501 & 508) |
| Complete: | 7 patients (502, 503, 504, 505, 506, 507, & 509) |

Dose administered in μg/kg/min over 8 hour infusion

| PATIENT | Cycle 1 | Cycle 2 | Cycle 3 | Cycle 4 | Cycle 5 | Cycle 6 | Cycle 7 | Cycle 8 |
|---|---|---|---|---|---|---|---|---|
| 501 | 50 | 75 | Nd | Nd | nd | nd | nd | Nd |
| 502 | 50 | 75 | 100 | 100 | 100 | 100 | 100 | 100 |
| 503 | 50 | 75 | 100 | 100 | 100 | 100 | 100 | 100 |
| 504 | 50 | 75 | 100 | 100 | 100 | 100 | 100 | 100 |
| 505 | 50 | 75 | 100 | 100 | 100 | 100 | 100 | 100 |
| 506 | 50 | 75 | 100 | 100 | 100 | 100 | nd | Nd |
| 507 | 50 | 75 | 100 | 100 | 100 | 100 | 100 | 100 |
| 508 | 50 | nd | Nd | Nd | nd | nd | nd | Nd |
| 509 | 50 | 75 | 100 | 100 | 100 | 100 | 100 | 100 |

*nd = not done

(b) (1) Narrative Summary of DMS Protocol #D0005 Study entitled:
“A Phase I Study of the Safety and Pharmacokinetics of Adenosine 5′-Triphosphate (ATP) When Administered b Intravenous Infusion on a Multiple Weekly Dose Schedule to Patients with Advanced Malignancies (Solid Tumors)”

As of Oct. 15, 2001, the above mentioned study has accrued nine patients that are represented by numbers 501-509. All but two patients have reached and tolerated 100 μg/kg/min of ATP for 8 hours. Patient 501 was withdrawn from the trial after two weeks without having attempted to highest dose of 100 μg/kg/min, which is given on the third week. Patient 508 requested to withdraw after one treatment without having attempted the highest dose of 100 μg/kg/min, which is given on the third week.

Patient 501—metastatic colon cancer with Type II diabetes mellitus. This patient was treated on weeks 1 and 2 after the second treatment (75 μg/kg/min) was withdrawn from the study because of the development of a deep vein thrombosis and evidence of progressive disease (serious clinical deterioration which was unrelated to test drug). During the two treatment weeks, the patient received 50 and 75 μg/kg/min of ATP infused over 8 hours with the development of grade 1 flushing and chest discomfort with a sensation of dyspnea-all toxicities rated as grade 1. His DVT was considered unrelated to the drug. This patient went onto to die in hospice from progressive disease.

Patient 502—metastatic mesothelioma. This patient tolerated the treatment on weeks 1 and 2 and was escalated to 100 μg/kg/min on week 3 and remained on this dose for weeks 4-8. During the infusion, but most markedly at the 100 μg/kg/min dose, he developed the following toxicities, grade 1 flushing and chest discomfort with a sensation of dyspnea (grade-I), nasal stuffiness and congestion and anxiety. On treatment weeks 5 and 7 the patient requested cessation of the ATP infusion (100 μg/kg/min) because of anxiety/nasal congestion without evidence of hypoxia. The infusion was stopped for 15-20 minutes and restarted and completed on each occasion. The patient had significant problems with local chest wall pain control secondary to disease, which required several modifications of his analgesic regimen during the study period. He also had grade I drowsiness, which his lorazepam dose reduced with good effect. The patient did comment on improved energy levels during the treatment period. Follow up on this patient showed that he died from progressive growth of his mesothelioma into his chest wall, the contralateral lung (left) and his scalp.

Patient 503—metastatic prostate cancer. This patient tolerated the treatment on weeks 1 and 2 and was escalated to 100 μg/kg/min on week 3 and remained on this dose for weeks 4-8. The patient completed his follow up. During treatment he commented on chest tightness and discomfort on several occasions—grade I. He had increased bone pain needing increased analgesia and had one episode of confusion related to too high a dose of lorazepam that cleared with drug withdrawal. He also developed a gradual decline in hemoglobin to 8.0 g/dl—grade II and for this was given a blood transfusion on 2 occasions. The anemia was felt to be due to several processes—bone marrow involvement by tumor, NSAID therapy and exaggerated by the blood taken for study pharmacokinetics and possibly, but less likely the ATP therapy. Following the second transfusion and stopping NSAIDs therapy the Hb stabilized. The patient commented on improved energy and appetite during ATP therapy. At the end of follow up, his PSA was unchanged compared to baseline.

Patient 504—metastatic breast cancer. This patient tolerated the treatment on weeks 1 and 2 and was escalated to 100 μg/kg/min on week 3 and remained on this dose for weeks 4-8. during treatment she developed a number of grade I toxicities—namely chest discomfort, nasal stuffiness (the latter often lasting several days post infusion), diarrhea, restless legs, and an episode of transient bradycardia with second degree AV block deteriorating to complete heart block which was asymptomatic and lasted approx. 10 seconds. (grade I—CTC criteria). She also had grade II nausea and vomiting (probably related to study drug) during one ATP treatment that was relieved by prophylactic anti-emetics. She completed the 8 weeks of ATP treatment and follow-up and was noted to have progressive disease and went back onto hormone therapy. The patient commented on increased energy and appetite with her weight increasing while on ATP treatment.

Patient 505—metastatic melanoma to scalp. This patient tolerated the treatment on weeks 1 and 2 and was escalated to 100 μg/kg/min on week 3 and remained on this dose for weeks 4-8. The toxicities noted were development of flushing, chest discomfort with a sensation of dyspnea and nasal stuffiness—all grade-I Grade I bradycardia with AV block (second degree AV block) degenerating to complete heart block with HR 35-40/min for no more than 10 seconds occurring on weeks 3 and 8, but not on weeks 4-7. On week 4 a repeat EKG revealed first degree AV block prior to the ATP infusion and this level of AV block did deteriorate during the ATP infusion. On week 5 she was in sinus rhythm. This patient was also receiving atenolol. The patient made subjective comments of improved energy and appetite while on study. The patient had progressive disease clinically and is now being considered for alternative biochemotherapy.

Patient 506—metastatic colon cancer with pulmonary and hepatic disease. This patient tolerated the ATP treatment on weeks 1 and 2 and was escalated to 100 μg/kg/min on week 3 and remained on this for week 4. the only toxicities noted were anxiety, headache and transient left sided chest ache-all rated grade I toxicities. She was admitted to hospital with fevers, worsening abdominal distension and increasing jaundice—which was felt to be due to progressive disease just before she was due to receive treatment 5. the patient was adamant in wanting to continue ATP therapy and that it made her feel “better” with more energy and improved appetite. She was continued on treatments 5 and 6 (at 100 μg/kg/min) and tolerated them without incident. Just before treatment 7 she was again admitted to hospital, this time with hepatic encephalopathy—bilirubin now 33.4 mg/dl and her ammonia was elevated (41 mg/dl). This patient was put on a terminal care regimen of comfort measures and died in hospital from progressive disease on Jul. 5, 2001.

Patient 507—metastatic prostate cancer. This patient tolerated the treatment on weeks 1 and 2 and was escalated to 100 μg/kg/min on week 3 and remained on this' dose for weeks 4-8. The patient developed grade I nasal stuffiness, chest discomfort, grade I nausea/vomiting on two occasions relieved by prophylactic anti-emetics (probably drug related) and grade-I constipation with a sensation of dyspnea (grade-I) on several occasions during the infusion-all of which have been short lasting/transient. He also has developed (grade II) increased pain in left clavicle, right side ribs and right leg, perineal numbness, and slight right hip flexor weakness, increased analgesic requirements which was felt most likely due to disease progression and not ATP related. Follow up of continuing pain was treated with anti-androgens and Samarium with good palliative response especially for back/leg pain.

Patient 508—prostate cancer. The patient was treated with the first infusion of ATP at 50 μg/kg/min and tolerated it very well. This patient was somewhat confused before starting ATP, which was noted at screening and did not appear to worsen during treatment. He withdrew from the study prior to week 2 therapy because he found the study to be tiring and exacting. No clinical toxicities were noted. Follow up-his worsening clinical condition, confusion (most likely related to CNS active drug or brain irradiation damage) caused him to go on to hospice care. Further information to be obtained from Dr. Fuselier PCP.

Patient 509—metastatic renal cell cancer. This patient tolerated dose escalation of ATP infusion from 50-100 μg/kg/min well. She completed the course of therapy from weeks 4-8, receiving ATP 100 μg/kg/min. Clinical toxicities during or peri ATP infusions were the following: Grade I anxiety, Grade I nausea and the patient vomited on several occasions, relieved by prophylactic anti-emetics, Grade I dyspepsia, and Grade I sinus-taschycardia. Prior to week 10 follow up, she developed Grade III confusion at home that lasted for 12-24 hours, possibly related to study drug or more likely related to analgesic drugs or progressive brain metastasis and radiation dementia. Most likely the Grade III confusion was the result of radiation brain injury plus analgesic drugs and probably not ATP related. At follow up on week 10: Grade III Confusion, Grade II Memory Loss, Grade II Anemia, Grade II Hypocalcemia, Grade I AST and Alk Phos elevation. The patient's condition improved by stopping analgesic drugs/increasing steroids. In retrospect her husband thinks that her confusion has been increasing somewhat over the last 2-3 months. All of these recent toxicities at follow up are considered unlikely to be related to ATP. She states subjective improvement in energy and activity. Further follow-upon week 12/13 remains to be done.

Overall, patients tolerated the ATP infusion well, with seven of nine patients tolerating the maximum allowable dose of 100 μg/kg/min, administered as a continuous 8 hour intravenous infusion. No grade II cardiac ischemia or grade III toxicities have been observed. Several patients have subjectively commented on improved appetite and energy during the study.

| | PATIENT No. | | | | |
|---|---|---|---|---|---|
| | 501 | 502 | 503 | 504 | 505 |
| Primary | Ca Colon | Mesothelioma | Prostate | Breast | Melanoma |
| Tumor | | | | | |
| ATP 8 hour | 50 & 75 μg/kg/min | 50, 75, & 100 | 50, 75, & 100 | 50, 75 & 100 μm/kg/min | 50, 75 & 100 μg/kg/min |
| infusion dose | | μg/kg/min | μg/kg/min | | |
| administered | | | | | |
| Duration on | Weeks 1&2 | Weeks 1-8 | Weeks 1-8 | Weeks 1-8 | Weeks 1-8 |
| Rx | | | | | |
| Dates on Rx | Jan. 23, 2001-Jan. | Jan. 24, 2001-Mar. | Feb. 23, 2001-Apr. | Mar. 22, 2001-May | Mar. 29, 2001-May |
| | 30, 2001 | 20, 2001 | 17, 2001 | 18, 2001 | 28, 2001 |
| Clin. | I Chest | I Chest D/Dysp | I Chest | I Chest | I Chest |
| Toxicity and | D/Dysp | I Flushing | D/Dysp | D/Dysp | D/Dysp |
| CTC-Grade | I Flushing | I Nasal | II Confusion - | I Flushing | I Nausea & |
| | | Congestion | 2° Drug | I Nasal | Vomiting |
| | | I Anxiety | induced | Congestion | I Flushing |
| | | II Chest wall | Anemia - see | I Anxiety | I Nasal |
| | | pain 2° disease | below | I AV | Congestion I |
| | | | | Block/CHB | AVBlock/CHB |
| | | | | II Nausea and | on 2 separate |
| | | | | Vomiting | treatments |
| | | | | I Diarrhea | |
| | | | | I Restless | |
| | | | | legs | |
| | | | | I Foot-Rash? | |
| Lab. | Nil noted | Nil noted | Hb 8.0 g/dl | Nil noted | Nil noted |
| Toxicity | Glucose | | decreased | | |
| | effect? | | Grade II - | | |
| | | | cause | | |
| Follow up | Progressive | Progressive | Alt Rx? | Going to | Progressive |
| | Disease. | disease. Died | Hormones + RXT | have | disease-will |
| | Died Mar. | May 16, 2001 | | hormone Rx | entere another |
| | 1, 2001 | | | | study |
| Tumor | Mass in | CXR, clinical | PSA Pre 4000 | CA 2729-99 | |
| burden | Rif/Jaundiced | new lesions | Post 3903 | iu/l | |
| Endpoints | | | Bone Scan- | | |
| | | | superscans X 2 | | |
| Other | | Felt more | Felt more | Feels more | Subjective |
| Endpoints | | energetic | energy during | energetic, | improvements |
| | | intermittintly | the study | appetite | in appetite and |
| | | during study | | increased, | energy level |
| | | | | weight | |
| | | | | increased | |
| | | | | improved | |
| | | | | vision | |

| | PATIENT No. | | | |
|---|---|---|---|---|
| | 506 | 507 | 508 | 509 |
| Primary | Ca Colon | Ca Prostate | Prostate | Renal cell |
| Tumor | | | | carcinoma |
| ATP 8 hour | 50 & 75 & | 50, 75 & 100 μg/kg/min | 50 μg/kg/min | 50, 75 & 100 |
| infusion dose | 100 μg/kg/min | | | μg/kg/min |
| administered | | | | |
| Duration on | Weeks 1-6 | Weeks 1-8 | Week 1 | Weeks 1-8 |
| Rx | | | | |
| Dates on Rx | May 8, 2001-Jun. | Jun. 1, 2001-Jul. | Jul. 24, 2001-Jul. | Aug. 21, 2001-Oct. |
| | 19, 2001 | 31, 2001 | 30, 2001 | 9, 2001 |
| Clin. | I Headache | I Chest | | I anxiety |
| Toxicity and | I Left sided | tightness/Dyspnea | | I nausea |
| CTC-Grade | chest ache | I Nasal stuffiness | | I vomiting |
| | | I nausea and | | I sinus |
| | | vomiting | | tachycardia |
| | | II increasing pain - | | |
| | | Progressive disease | | |
| Lab. | Increasing | Nil noted | Nil noted | At FU grade I |
| Toxicity | bilirubin | | | AST/Alk Phos |
| | and LFT's - | | | increase |
| | disease | | | |
| | progression | | | |
| Follow up | Died July | Week 10 - more | Patient | Episode of |
| | 5^{th} 2001 | pain. Rx with | withdrew | confusion Gr 3, |
| | | hormones and | from study. | at week 10. |
| | | Samarium | Also had | Probably not |
| | | improved by week | worsening | ATP related |
| | | 12/13 | performance | |
| | | | status and | |
| | | | confusion | |
| Tumor | | PSA unchanged | Not | MRI multiple |
| burden | | but increased after | applicable | cra mets - end of |
| Endpoints | | stopping Rx | | study radiation |
| | | | | necrosis rather |
| | | | | than mets? |
| Other | Felt more | Felt more energy | Not | Subjective |
| Endpoints | energy & | while on ATP Rx | applicable | improvement an |
| | activity | | | energy/activity |
| | while on | | | |
| | ATP Rx | | | |

(b) (2, 3, 4) Summary of Safety Reports

Medwatch Reports/Serious Adverse Events

| REPORT | PATIENT | SERIOUS ADVERSE EVENT | |
|---|---|---|---|
| DATE | ID | AND CAUSE | DATE OF EVENT |
| Feb. 16, 2001 | 501 | DVT; unrelated to study drug | Feb. 1, 2001 |
| Jun. 22, 2001 | 501 | Death from progressive disease; | Mar. 1, 2001 |
| | | unrelated to study | |
| | 502 | Hospitalization for Pain Control; | Jun. 8, 2001 |
| | | unrelated to drug study | |
| | 502 | Death from progressive disease; | May 16, 2001 |
| | | unrelated to study (off study) | |
| | 506 | Hospitalization due to progressive | Jun. 4, 2001 |
| | | disease; unrelated to study | |
| Jul. 6, 2001 | 502 | Follow up report with corrected date | Apr. 18, 2001 |
| | 506 | Death from progressive disease; | Jul. 5, 2001 |
| | | unrelated to study | |

DEATHS

| | | | | RELATION TO |
|---|---|---|---|---|
| PATIENT | TIME ON STUDY | DATE | CAUSE | STUDY |
| 501 | Jan. 23-Jan. 30, 2001 | Mar. 1, 2001 | Progressive disease | Unrelated to drug |
| | (2 cycles) | | | study |
| 502 | Jan. 30-Mar. 20, 2001 | May 16, 2001 | Progressive disease | Unrelated to drug |
| | (8 cycles) | | | study |
| 506 | May 8-Jun. 19, 2001 | Jul. 5, 2001 | Progressive disease | Unrelated to drug |
| | (6 cycles) | | | study |
| 503 | Feb. 23-Apr. 17, 2001 | Jul. 13, 2001 | Progressive disease | Unrelated to drug |
| | (8 cycles) | | | study |

(b) (5)
Overall, the nine qualified patients tolerated the ATP infusion well. The seven evaluable patients tolerated the maximum allowable dose of 100 μg/kg/min, administered as a continuous 8 hour intravenous infusion.
No grade II cardiac ischemia or grade III toxicities have been observed.
Several patients have subjectively commented on improved appetite and energy during the study.
Based on the experience of the P.I. with over 40 infusion cycles, the minimum time of EKG/physiological monitoring of at least 2 hours post end of ATP infusion has been changed to at least 1 hour. Both the physicians and patients are more comfortable and this provides better convenience to the patient.
We have seen in patient 506 that ATP is well tolerated in terminal patients.

Interim analysis of clinical data of patients 502-505, which are the first four evaluable patients, has been performed. The following highlights were noted:
Increases in Global Health Status of the QOL EORTC QLQ-C30 from screening (week <1) to follow-up (week 10) with a decline at week 13 follow-up.
Decreases in appetite loss for all four patients from screening to week 10 (follow-up) with a return to screening baseline at week 13 follow-up.
Consistent decreases in SGOT (serum glutamate oxaloacetate transaminase), SGPT (serum glutamate pyruvate transaminase) and serum LDH (lactate dehydrogenase) for all four patients from week 1 (pre-dose) to week 13 (follow-up).
Stabilization of body weight from week 1 pre-dose to week 13 follow-up.
Small increases in Karnofsky Performance Status from week 1 pre-dose to week 13 follow-up.
Small increase in Skeletal Muscle Strength (voluntary) from screening (week <1) to week 8 (last infusion cycle).

The pharmacokinetics of ATP performed on weeks (cycles) 1, 3 and 8 demonstrated the following for patients 502-505:
Increases in total blood ATP pools from time 0 to 8 hours (end of the infusions) of about 70-90% with a decline to baseline (time 0) at 24 hours.
The average increases in Initial ATP Release Rates (from Red Blood Cells) from time 0 to 8 hours were about 200-400% with comparable increases in extracellular (blood plasma) ATP pools.
   (b) (6) Not applicable. There have been no pre-clinical studies performed during this time period.
   (b) (7) 15 Month Stability Report

The 15 months stability testing of the refrigerated ATP (5° C.) showed a 99.7% of label. See attached AAI International Analytical Testing Report Aug. 2, 2001.

(c) Not applicable. There have been no changes in the Investigational Plan to replace the previous year.

(d) Not applicable. There have been no Investigator Brochure revisions.

Phase I Modification/Amendments:

| AMENDMENTS | DATE | FORM FDA 1571 SECTION 11. | CONTENTS |
|---|---|---|---|
| 001 | Jul. 11, 2000 | CMC Information | 1. Stability data to support 10, 30 |
| | | Response to FDA request for | & 96 hrs room temperature in an |
| | | information | infusion bag |
| | | | 2. 12 hr. expiration date set for |
| | | | diluted ATP |
| | | | 3. Stability protocol conforms to |
| | | | ICH guidelines |
| | | | 4. Statement added to pg 5-4 of |
| | | | IND “the final filled container |
| | | | passed the U.S. Pharmacopoeia |
| | | | membrane method sterility test.” |
| | | | 5. We commit to LAL specs., |
| | | | report results of stability testing, |
| | | | evaluate ATP impurity limit |
| | | | (10%) at 12 month time pt. and |
| | | | consider tightening. |
| 002 | Aug. 7, 2000 | Clinical Information | 1. Clarify SAE reaction that |
| | | Response to FDA request for | would result in removal from trial. |
| | | information | 2. Decreasing infusion rate if |
| | | | reaction rather than discontinuing. |
| | | | 3. Rationale for start dose at 50 mcg/kg/min |
| | | | vs. 25 mcg/kg/min. |
| | | | 4. Exclusion criteria of ischemic |
| | | | cardiac disease, CHF, SSS 2^{nd}, 3^{rd}, |
| | | | AV block added. |
| 003 | Sep. 29, 2000 | Change in Protocol | 1. Revised clinical protocol dated |
| | | CMC Information | Jul. 31, 2001: |
| | | | Appendix F F-1 pain scale |
| | | | Page 5. Adverse events |
| | | | Page 6. Table. Add 13 week |
| | | | follow up visit, and clarification. |
| | | | Page 8 Exclusion criteria of |
| | | | ischemic cardiac disease, CHF, |
| | | | SSS 2^{nd}, 3^{rd}, AV block added. |
| | | | Page 9 Follow up visit week 13 |
| | | | added. |
| | | | Page 10 & 11 Decreasing |
| | | | infusion rate if reaction rather than |
| | | | discontinuing if an adverse event |
| | | | occurs. If the DLT occurs on week |
| | | | 1, 3 or 8 then no pk samples taken. |
| | | | Page 16 Assessment of tumor |
| | | | response based on RECIST |
| | | | criteria |
| | | | Page 19 Clarity on definitions |
| | | | of adverse events using NCI CTC |
| | | | version 2.0 |
| | | | Page 22 Patient withdrawal |
| | | | clarification |
| | | | 2. IRB approval for revised |
| | | | clinical protocol |
| | | | 3. 3 month stability data: ATP |
| | | | remains stable at 3 months under |
| | | | routine storage conditions. |
| 004 | Jan. 18, 2000 | CMC Information | 1. Recommended storage on label |
| | | Response to FDA request for | 2. Drug substance testing |
| | | information | according to USP methods |
| | | | 3. Test method for pyrogen testing |
| | | | indicated: pyrogen free |
| | | | 4. Reference standard source and |
| | | | C of A provided |
| | | | 5. Quantitative test for color & |
| | | | USP Particulate Matter for |
| | | | injection test to be done on next |
| | | | lot of ATP |
| 005 | May 1, 2001 | CMC Information | 1. 12 month stability report |
| | | Response to FDA request for | provided |
| | | information | |
| 006 | Jun. 22, 2001 | Change in Protocol | 1. Additional 2 PK samples: pre- |
| | | CMC Information | dose & post infusion & removal of |
| | | Response to FDA request for information Info | 8.25 hour sample from week 1, 3, |
| | | Safety Reports | 8 for overall less 5 ml blood draw. |
| | | | 2. Reducing minimum |
| | | | physiological monitoring time to |
| | | | at least 1 hr. |
| | | | 3. Safety Reports for patients 501, |
| | | | 502, & 506 |
| 007 | Jul. 6, 2001 | Safety Reports | Safety reports for patients 502 & |
| | | | 506 |

(e) New Amendment
Amendment 008

We would like to introduce an amendment to IND #60.517, DMS Protocol #D0005 “A Phase I Study of the Safety and Pharmacokinetics of Adenosine 5′-Triphosphate (ATP) When Administered by Intravenous Infusion on a Multiple Weekly Dose Schedule to Patients with Advanced Malignancies (Solid Tumors)”.

The present protocol has a secondary objective to evaluate the effects of ATP treatment on Cancer Cachexia utilizing Quality of Life parameters. Three of these parameters are EORTC QLQ-C30 patient-oriented questionnaire, skeletal (voluntary) muscle strength and percent body fat. Patient evaluations are currently performed on the screening visit and at weeks 2, 4, 8 (before ATP infusions), and at follow-up visits on weeks 10, and 13. The screening value is used as the patients' baseline. In some patients a deterioration in overall quality of life between screening and week 1, prior to the administration of study drug has been observed. Initial date for the first four evaluable patients (patients 502-505), demonstrate a drop in Karnofsky Performance Status—for two patients (502, 504) between screening and week 1 prior to dosing. Thus potentially underestimating the efficacy of ATP treatment in improving these three cachexia and quality of life parameters. A more accurate baseline for evaluation of the efficacy of ATP treatment on cancer cachexia and quality of life parameters is therefore recommended.

The protocol amendment we are proposing will add week 1 determinations of EORTC QLQ-C30 questionnaire, skeletal (voluntary) muscle strength and percent body fat for the purpose of improving the accuracy of the baseline comparison.

Attached is a revised protocol dated Oct. 15, 2001 highlighting the additions and changes on pages 1, 6, 10 (Amendment 006), and 13.

### Discussion of Study Design, Including the Choice of Control Groups

This Phase I study had an open design, with no control groups. This early stage trial was designed to provide clarity about safety at the dosing tested, and indications of efficacy, so the design and the small patient number were considered to be appropriate for this type of study.

Selection of Study Population

Inclusion Criteria

The study included 24 adult patients with histologically/cytologically confirmed advanced malignancies (solid tumors) not curable by conventional therapies who fulfilled the following eligibility requirements:
1. Preferably had measurable disease but this was not mandatory.
2. Karnofsky performance status ≧60.
3. Life expectancy ≧12 weeks.
4. At least 3 weeks lapsed time since prior chemotherapy or radiation therapy, with myelosuppression from the prior therapy reversed (≧6 weeks for prior nitrosoureas and mitomycin C).
5. No other investigational therapy within 30 days of entering this study.
6. Adequate hepatic, renal, and bone marrow function as defined by:
   a. White blood cell count ≧3,500 per mm³
   b. Absolute neutrophil count (ANC) ≧2,000 per mm³
   c. Platelet count ≦100,000 per mm³
   d. Serum creatinine ≦1.5 mg/100 mL and estimated creatinine clearance (CrCL) (Cockroft-Gault) >60 mL/min
   e. Blood urea nitrogen ≦25 mg/100 mL
   f. Serum glutamic-oxaloacetic transaminase (SGOT) ≦3 times normal
   g. Serum glutamate pyruvate transaminase (SGPT) ≦3 times normal
   h. Serum bilirubin ≦2.0 mg/100 mL
7. Adequate cardiovascular function as determined by lack of clinical evidence of congestive heart failure, angina pectoris, and/or significant arrhythmia, and no myocardial infarction within the past 6 months (by clinical history).
8. Adequate pulmonary function as defined by:
   a. No clinical evidence of acute chronic obstructive pulmonary disease (COPD)
   b. Forced expiratory volume in one second (FEV₁) ≧50% of predicted value
   c. Arterial oxygen tension, at rest and breathing room air as measured by pulse oximetry, was ≧90%.
9. Brain metastases in patients with advanced refractory cancers were adequately controlled with radiotherapy.
10. Women of childbearing potential were taking adequate medical contraceptive measures.
11. Over 18 years of age.
12. Written informed consent form signed prior to study entry.

Exclusion Criteria
1. History of asthma, or known to exhibit >20% reversibility in FEV₁ following albuterol administration.
2. History of clinically significant ischemic cardiac disease (currently under treatment), congestive heart failure (New York Heart Association grade 3 or 4), or evidence of clinically significant conduction system disease in the absence of a pacemaker (sick sinus syndrome, 2^{nd} or 3^{rd} degree artioventricular (AV) block).
3. Ongoing long-term treatment with theophylline or dipyridamole (or dipyridamole/aspirin). Patients were included in the trial if they had discontinued such treatment for at least 14 days.
4. Prior history of severe adverse reaction (CVS/R/S) to adenosine.
5. Receiving maintenance antianginal drug therapy.
6. Uncontrolled medical illness that precluded completion of the study protocol.
7. Average daily pain scores >5 on a simple VAS pain assessment (0-10) scale.

### Removal of Patients from Therapy or Assessment

Subjects could withdraw from the study at any time for any reason.

Subjects could have been withdrawn from this trial by the principal investigator(s) at any time for the following reasons:

Serious clinical deterioration, which was unrelated to test drug administration, or Any grade 3 or greater toxicity (or grade 2 or greater cardiac ischemia) attributable to the ATP infusion as per National Cancer Institute Common Toxicity Criteria (CTC) version 2, or

Patients experiencing grade 4 pulmonary toxicity attributed to ATP persisting >20 minutes after reduction of ATP infusion to the next lowest dose tier or requiring two reductions in ATP dose tier without improvement to grade 1 toxicity.

In all cases of patient withdrawal, the reasons for withdrawal and outcome in these subjects were fully documented. A follow-up visit was scheduled at 30 days after withdrawal and due diligence was exercised in obtaining as much designated study information as possible.

Treatments

Treatments of Administered

ATP was administered to each subject once weekly for 8 consecutive weeks as an 8 hour intravenous infusion at rates of 25, 50, 75, or 100 μg/kg/min. Preparation of the infusion solution required that the volume of one vial of ATP be aseptically removed using a syringe and added to a 250 mL bag of 0.5% normal saline (0.45% NaCl) (volume adjusted by removal of 20 mL of saline to compensate for the addition of 20 mL ATP). The concentration of the final sterile solution was 8 mg/mL ATP. The solution was administered by continuous intravenous infusions using an Ivac or similar infusion device through venous access in a peripheral vein. If venous access was a problem, either a Hickman catheter or its equivalent, or an Infusaport or its equivalent was inserted to provide vascular access.

Identity of Investigational Product

Adenosine 5′-triphospate (ATP) was provided as a sterile solution in single use vials. Each vial contained 2 grams of ATP as a sodium salt in 20 mL of Water for Injection, at pH 6.7-7.2. The concentration of ATP in the vials was 100 mg/mL. Storage of the clinical solutions was at a controlled refrigerated temperature (2-8° C.). The lot number was 0303976.

Method of Assigning Patients to Treatment Groups

Each patient received doses of ATP of 50-100 μg/kg/min dose. The 50 μg/kg/min dose could be reduced to 25 μg/kg/min if adverse effects occurred. The rate of infusion of ATP during each of the weekly 8 hour infusions was determined according to the following set of guidelines:
1. In all patients, the initial 8 hour infusion (infusion 1 on Day 0, Week 1) was at 50 μg/kg/min a rate of infusion that has been generally well tolerated in other clinical studies in cancer patients (16).
2. If adverse effects did not intervene, the rate of infusion in Week 2 was increased to 75 μg/kg/min (infusion 2). If well tolerated, the dose was increased in Week 3 to 100 μg/kg/min. Again, if well tolerated, this infusion rate was administered for the remaining five infusions (infusions 4-8).
3. If adverse events occurred in association with any infusion at 50, 75, or 100 μg/kg/min, administration of the ATP infusion was to have been decreased to the next lower infusion rate or stopped to allow for treatment-related complications to subside. If the infusion was stopped, the patient could restart the treatment at the next lower infusion dose. Succeeding infusions in that patient were at the infusion dose tolerated at the end of the previous treatment. If adverse events occurred in patients being infused at 25 μg/kg/min, the infusions were terminated and the patient received no additional infusions.

The physician's decisions about the infusions and infusion dosing were guided by the following criteria:
An infusion was stopped at any time when requested by the patient.
An infusion was stopped by the physician at the occurrence of any grade 3 or greater toxicity or any grade 2 cardiac ischemia demonstrated by electrocardiography (criteria defined by the National Cancer Institute, CTC version 2).
The patient may have continued the ATP infusion at one dose level below that causing grade 3 or greater toxicity or grade 2 cardiac ischemia as above, providing the patient and physician agreed to proceed. No patient received <25 μg/kg/min.
Any patient who required two consecutive ATP infusion dose reductions was considered to have a dose limiting toxicity (DLT) and was removed from the study.

### Selection of Doses in the Study

Infusing ATP into cancer patients at or near maximally tolerated dose rates over extended time periods (30-96 hr every 2-4 weeks) have been achieved. These experiences have shown that dose rates of ATP at ≦100 μg/kg/min are relatively safe and have suggested that ATP can inhibit the development of cancer cachexia in such patients (15,16). Given the greater acceptance of shorter infusion times in an outpatient setting, the present study was developed to determine whether patients would tolerate infusions at the same rates but given for 8 hours, once a week, i.e., a lower total dose per treatment but given more frequently.

### Selection and Timing of Dose for Each Patient

The ATP infusion was started between 0700 and 1100 hours on each day of treatment. For information about dose selection for each patient, see section 9.4.3.

Blinding

This study was not blinded.

Prior and Concomitant Therapy

Use of substances that may have potentiated or inhibited the activity of adenosine (e.g., caffeine-containing foods such as chocolate, Coke, Pepsi, coffee, tea, or drugs such as theophylline, dipyridamole, and papaverine) were to be avoided for 12 hours before the start of the infusion, during infusion, and for 24 hours after the end of the infusion.

Treatment Compliance

Study drug was administered by the site staff. Compliance in this study was confirmed by the treatment administration records kept for each study drug infusion in the patient's case report form (CRF).

Efficacy and Safety Variables

Schedule of Assessments

The following table outlines the assessments that were conducted at each study visit.

Phase I Safety & Pharmacokinetics of ATP in Advanced Solid Tumors (#D00005.04) ATP Therapeutics, Inc. Final—Sep. 21, 2005

TABLE 1
Schedule of Assessments

| | Week a | | | | | | | | | |
|---|---|---|---|---|---|---|---|---|---|---|
| | | | | | | | | | 8 | 10 & 13 |
| | <1 | 1 | 2 | 3 | 4 | 5 | 6 | 7 | Infusion | Follow-up |
| Procedures | Screening | Infusion 1 | Infusion 2 | Infusion 3 | Infusion 4 | Infusion 5 | Infusion 6 | Infusion 7 | 8 | visits |
| Recruitment and | X | | | | | | | | | |
| Signed Consent | | | | | | | | | | |
| History of Recent | X | | | | | | | | | |
| Weight Loss | | | | | | | | | | |
| Complete Medical | X | | | | | | | | | |
| History | | | | | | | | | | |
| Complete Physical | X | | | | | | | | | X |
| Exam | | | | | | | | | | |
| 12-Lead | X | | | | | | | | | X |
| Electrocardiogram | | | | | | | | | | |
| Chest X-ray | X | | | | | | | | | |
| Pulmonary | X | | | | | | | | | |
| Function Test | | | | | | | | | | |
| Resting Pulse | X | | | | | | | | | |
| Oximetery SaO₂ | | | | | | | | | | |
| Tumor Status | X | | | | | | | | | |
| Percent Body Fat | X | X | X | | X | | | | X | X |
| Body Weight | X | X | X | X | X | X | X | X | X | |
| QOL Evaluation | X | X | X | | X | | | | X | |
| Skeletal Muscle | X | X | X | | X | | | | X | X |
| Strength | | | | | | | | | | |
| Radiological | X^{b} | | | | | | | | | X |
| Tumor Imaging | | | | | | | | | | |
| Clinical Chemistry | X | X | X | | X | | X | | X | X |
| and Cachexia | | | | | | | | | | |
| Markers | | | | | | | | | | |
| Hematology | X | X | X | | X | | X | | X | X |
| Urinalysis | X | X | X | | X | | X | | X | X |
| Blood for ATP | | X^{c} | X^{d} | X | | | | | X | |
| Pharmacokinetics | | | | | | | | | | |
| Tumor-related | X | X | X | X | X | X | X | X | X | X |
| Symptoms | | | | | | | | | | |
| Concurrent | X | X | X | X | X | X | X | X | X | X |
| Medication | | | | | | | | | | |
| Karnofsky | X | X | X | X | X | X | X | X | X | X |
| Performance Status | | | | | | | | | | |
| Focused Physical | | X | X | X | X | X | X | X | X | |
| Exam | | | | | | | | | | |
| Adverse | | X | X | X | X | X | X | X | X | X |
| Experience | | | | | | | | | | |
| Assessment | | | | | | | | | | |
| Monitoring of Vital | | X | X | X | X | X | X | X | X | |
| Signs and ECG | | | | | | | | | | |
| Before, During and | | | | | | | | | | |
| After Infusion | | | | | | | | | | |

^{a}Day of first infusion was day 0 of week 1
^{b}Performed up to 28 days before study entry
^{c}In patients experiencing dose limiting toxicity, where infusion rates were decreased by 25 μg/kg/min at weeks 1, 2, 3, or 8, pharmacokinetic analyses were not performed.
^{d}On the second administration of ATP (Week 2, day 0), ATP pharmacokinetic samples were only obtained preinfusion (time 0) and just before the end of the infusion (8 h).

Study Patients

Disposition of Patients

A total of 15 patients were enrolled into the study and 7 patients completed it (Table 14.1.1). Three patients withdrew from the study at their request and five patients were withdrawn by the investigator due to serious clinical deterioration (SCD). Individual patient listings of discontinuations are provided in Appendix Listing 16.2.2.

Three patients did not meet the inclusion criterion for adequate hepatic, renal, and bone marrow function (#504, 506, 513), but were granted an exception, along with patient #509, due to laboratory results that were deemed to be not clinically significant (Appendix Listing 16.2.1.1). These patients were therefore allowed to enter the study.

Protocol Deviations

Eight patients had violations in study drug administration (Table 14.1.2). Six incidents of taking substances that were to be avoided occurred in 5 patients (appendix Listing 16.2.5.1).

Study Population Results

Data Sets Analyzed

Two data sets were analyzed: the safety sample, which included all patients who received at least one infusion of study medication, and the efficacy sample which included all patients who received Weeks 1 to 3 of ATP. The safety sample and the intent-to-treat (ITT) sample were identical. All 15 patients were included in the (ITT) sample, and 13 patients were included in the efficacy sample) (Table 14.1.1-14.1.2). The efficacy sample will be used for the quality of life and tumor response analyses.

### Demographic and Other Baseline Characteristics

Demographic and baseline characteristics are summarized in the table below. Patients in the study tended to be older (mean age 60.6 years). Slightly more males than females were included (60.0%). All patients were Caucasian. The patient demographics are summarized in more detail in Table 14.1.3. Individual patient, demographic data are provided in Appendix Listing 16.2.3.1.

At the time of diagnosis, most patients had stage III or IV cancer (78.6%) with secondary tumors (93.3%) (Table 14.1.4.1). A mean of approximately 3.5 years had elapsed since their cancer was first diagnosed. Most patients had had cancer-related surgery (80.0%) and chemotherapy (93.3%). Individual patient data for previous cancer-related surgery, radiation therapy, and chemotherapy are provided in Appendix Listings 16.2.4.3-5.

TABLE 6
Demographics and Baseline Characteristics

| Characteristic | N = 15 |
|---|---|
| Mean age (years) | 60.6 |
| Minimum-Maximum | 38.0-80.0 |
| Gender n (%) | |
| Male | 9 (60.0) |
| Female | 6 (40.0) |
| Race n (%) | |
| Caucasian (not Hispanic origin) | 15 (100.0) |
| Stage of cancer at time of diagnosis n (%) | |
| I | 1 (7.1) |
| II | 2 (14.3) |
| III | 4 (28.6) |
| IV | 7 (50.0) |
| Missing | 1 (7.1) |
| Secondary tumors n (%) | 14 (93.3) |
| Mean time since cancer diagnosed (months) | 41.9 |
| Mean time since staging performed (months) | 40.7 |
| Previous cancer treatment n (%) | |
| Surgery | 12 (80.0) |
| Radiation therapy | 7 (46.7) |
| Chemotherapy | 14 (93.3) |

CT results are summarized in Table 14.1.4.2. All patients who had CT body scans had abnormalities (46.7%). Brain scans were not done in most patients. Individual patient data for tumor imaging are provided in Appendix Listing 16.2.4.2.

The pre-study medical and surgical history findings are presented in Appendix Listings 16.2.3.2 and 16.2.3.3, respectively

ATP Therapeutics, Inc.

Protocol #DMS D0005: A Phase I Study of ATP in Advanced Cancer

APPENDIX 16.2.4.1
Listing of Pre-Study Oncological Diagnosis

| | | When was | | | | | | |
|---|---|---|---|---|---|---|---|---|
| | | Cancer First | Location of | | | Date of Stage | | |
| | | Diagnosed? | the Primary | Histological/Cytological | Stage of | Performing | Secondary | If yes, specify: |
| Site | Patient | (mm/dd/yyyy) | Tumor | Type of Tumor | Cancer | (mm/dd/yyyy) | Tumors? | Location |
| 001 | 501 | 04/08/1999 | COLON | ADENOCARCINOMA | IV | 04/08/1999 | Yes | LIVER, SPLEEN, |
| | | | | | | | | PERITONEAL |
| | | | | | | | | CARCINOMATOSIS |
| | 502 | 12/04/1998 | LUNG | MESOTHELIOMA | 2 | 12/04/1998 | No | |
| | 503 | —/—/1998 | PROSTATE | ADERO CA | UNK | —/—/1998 | Yes | BONE METS |
| | 504 | 08/30/1985 | BREAST | INFILTRATING | I | 09/03/1985 | Yes | BONE |
| | | | | DUCTAL CA | | | | |
| | 505 | 07/31/2000 | SCALP | MELANOMA | III | 07/31/2000 | Yes | SKIN LESIONS |
| | 506 | 01/18/1999 | COLON | ADENO CA | 4 | 01/21/1999 | Yes | LUNG, LIVER |
| | 507 | 10/22/1997 | PROSTATE | ADENOCARCINOMA | III | 10/22/1997 | Yes | BONE |
| | 508 | 05/27/1997 | PROSTATE | ADENOCARCINOMA | III | 05/27/1997 | Yes | BRAIN, BONE |
| | 509 | 09/05/2000 | RENAL | RENAL CELL | 4 | 09/05/2000 | Yes | LUNG, BRAIN |
| | 510 | 05/18/1992 | PROSTATE | ADENO | IIB | 05/18/1992 | Yes | BONE |
| | 511 | 12/20/1999 | PENIS | SQUAMOUS CELL | III | 05/22/2000 | Yes | PELVIS |
| | 512 | 07/27/2001 | ADRENAL | ADRENOCORTICAL | IV | 07/27/2001 | Yes | RETROPERITONEUM, |
| | | | CA. | | | | | LIVER & LUNGS |
| | 513 | —/—/1999 | PROSTATE | ADENOCA & RENAL | 4 | —/—/2000 | Yes | BONE |
| | | | & RENAL | CELL | | | | |
| | | | CELL | | | | | |
| | 514 | 08/27/2001 | COLON | ADENOCARCINOMA | 4 | 08/27/2001 | Yes | Liver |
| | 515 | 11/23/1999 | COLON | ADENOCARCINOMA | 4 | 11/23/1999 | Yes | Omentum (resected |
| | | | | | | | | 1999), ABD. Mass, |
| | | | | | | | | parraortic adenopathy |

ATP Therapeutics, Inc.

Protocol #DMS D0005: A Phase I Study of ATP in Advanced Cancer

TABLE 14.2.11
Summary of ATP Pharmacokinetics
ITT Sample

| Visit | Time Point | | N | Mean | Std. Dev. | Median | Minimum | Maximum |
|---|---|---|---|---|---|---|---|---|
| ATP Level (mM) | | | | | | | | |
| Week 1 (infusion 1) | Time ‘0’ (pre-infusion) | | 15 | 0.615 | 0.2292 | 0.550 | 0.310 | 1.100 |
| | 4 | hours into infusion | 14 | 0.808 | 0.3422 | 0.745 | 0.400 | 1.500 |
| | 8 | hours (before the end) | 15 | 0.896 | 0.3267 | 0.770 | 0.580 | 1.600 |
| | 8.25 | hours | 1 | 0.860 | 0.0000 | 0.860 | 0.860 | 0.860 |
| | 8.5 | hours | 15 | 0.895 | 0.3012 | 0.810 | 0.560 | 1.520 |
| | 9 | hours | 14 | 0.912 | 0.4413 | 0.765 | 0.520 | 1.900 |
| | 10 | hours | 14 | 0.826 | 0.3241 | 0.735 | 0.460 | 1.500 |
| | 11 | hours | 15 | 0.828 | 0.4221 | 0.680 | 0.460 | 2.000 |
| | 12 | hours | 15 | 0.845 | 0.3988 | 0.690 | 0.480 | 1.800 |
| | 14 | hours | 11 | 0.857 | 0.4214 | 0.730 | 0.460 | 1.700 |
| | 16 | hours | 10 | 0.905 | 0.4483 | 0.755 | 0.480 | 1.800 |
| | 20 | hours | 11 | 0.772 | 0.2492 | 0.670 | 0.490 | 1.200 |
| | 24 | hours | 15 | 0.715 | 0.2618 | 0.620 | 0.440 | 1.400 |
| Week 3 (Infusion 3) | Time ‘0’ (pre-infusion) | | 12 | 0.610 | 0.2487 | 0.560 | 0.240 | 1.000 |
| | 4 | hours into infusion | 11 | 0.843 | 0.4953 | 0.540 | 0.350 | 1.660 |
| | 8 | hours before the end) | 11 | 0.955 | 0.4570 | 0.770 | 0.490 | 1.600 |
| | 8.25 | hours | 0 | | | | | |
| | 8.5 | hours | 11 | 0.989 | 0.4495 | 0.960 | 0.400 | 1.660 |
| | 9 | hours | 11 | 0.908 | 0.3940 | 0.950 | 0.400 | 1.600 |
| | 10 | hours | 10 | 0.906 | 0.3903 | 1.000 | 0.450 | 1.600 |
| | 11 | hours | 11 | 0.885 | 0.5165 | 0.670 | 0.410 | 1.840 |
| | 12 | hours | 11 | 0.903 | 0.5340 | 0.820 | 0.360 | 2.100 |
| | 14 | hours | 9 | 0.853 | 0.4849 | 0.600 | 0.400 | 1.700 |
| | 16 | hours | 9 | 0.859 | 0.4107 | 0.740 | 0.360 | 1.700 |
| | 20 | hours | 8 | 0.851 | 0.6012 | 0.610 | 0.520 | 2.300 |
| | 24 | hours | 11 | 0.687 | 0.3312 | 0.580 | 0.330 | 1.500 |
| Week 8 (infusion 8) | Time ‘0’ (pre-infusion) | | 6 | 0.573 | 0.1643 | 0.560 | 0.360 | 0.810 |
| | 4 | hours into infusion | 5 | 0.702 | 0.1438 | 0.770 | 0.530 | 0.860 |
| | 8 | hours (before the end) | 5 | 0.826 | 0.2401 | 0.890 | 0.530 | 1.060 |
| | 8.25 | hours | 0 | | | | | |
| | 8.5 | hours | 3 | 1.000 | 0.3504 | 0.980 | 0.660 | 1.360 |
| | 9 | hours | 4 | 1.030 | 0.3492 | 1.155 | 0.530 | 1.280 |
| | 10 | hours | 4 | 0.963 | 0.3051 | 1.080 | 0.520 | 1.170 |
| | 11 | hours | 4 | 0.973 | 0.3298 | 1.010 | 0.550 | 1.320 |
| | 12 | hours | 4 | 0.925 | 0.2989 | 1.050 | 0.480 | 1.120 |
| | 14 | hours | 5 | 0.980 | 0.3787 | 0.950 | 0.480 | 1.520 |
| | 16 | hours | 5 | 0.894 | 0.4669 | 0.700 | 0.490 | 1.690 |
| | 20 | hours | 5 | 0.960 | 0.3940 | 0.900 | 0.500 | 1.580 |
| | 24 | hours | 5 | 0.708 | 0.1016 | 0.750 | 0.550 | 0.810 |

ATP Therapeutics, Inc.

Protocol # DMS D0005: A Phase I Study of ATP in Advanced Cancer

TABLE 14.2.11
Summary of ATP Pharmacokinetics
ITT Sample

| Visit | Time Point | | N | Mean | Std. Dev. | Median | Minimum | Maximum |
|---|---|---|---|---|---|---|---|---|
| Initial ATP Release | Time ‘0’ (pre-infusion) | | 13 | 15.469 | 13.4081 | 10.100 | 2.900 | 46.900 |
| Rates (nM/min) | 4 | hours into infusion | 13 | 33.407 | 33.7255 | 23.400 | 1.600 | 134.300 |
| Week 1 (Infusion 1) | 8 | hours (before the end) | 13 | 47.224 | 34.5186 | 31.750 | 9.250 | 124.900 |
| | 8.25 | hours | 1 | 10.400 | 0.0000 | 10.400 | 10.400 | 10.400 |
| | 8.5 | hours | 12 | 41.353 | 30.3795 | 32.420 | 7.900 | 90.100 |
| | 9 | hours | 13 | 25.408 | 17.1052 | 26.400 | 3.400 | 57.600 |
| | 10 | hours | 13 | 13.959 | 9.4173 | 10.400 | 4.000 | 36.690 |
| | 11 | hours | 15 | 14.743 | 11.8121 | 9.700 | 2.330 | 45.500 |
| | 12 | hours | 15 | 12.565 | 9.7407 | 10.200 | 3.200 | 36.110 |
| | 14 | hours | 9 | 12.067 | 14.6425 | 7.140 | 1.500 | 47.460 |
| | 16 | hours | 10 | 15.331 | 16.6991 | 10.200 | 4.500 | 61.800 |
| | 20 | hours | 9 | 20.063 | 18.3352 | 12.620 | 5.400 | 57.900 |
| | 24 | hours | 13 | 17.452 | 10.3874 | 14.800 | 4.500 | 37.800 |
| Week 3 (Infusion 3) | Time ‘0’ (pre-infusion) | | 10 | 18.228 | 11.6532 | 13.350 | 5.740 | 44.340 |
| | 4 | hours into infusion | 9 | 55.414 | 79.9132 | 35.100 | 5.800 | 265.200 |
| | 8 | hours (before the end) | 9 | 48.283 | 24.6234 | 49.000 | 13.600 | 96.000 |
| | 8.25 | hours | 1 | 13.400 | 0.0000 | 13.400 | 13.400 | 13.400 |
| | 8.5 | hours | 10 | 38.780 | 21.0016 | 39.500 | 9.300 | 72.000 |
| | 9 | hours | 9 | 34.992 | 23.0158 | 37.330 | 6.900 | 69.500 |
| | 10 | hours | 9 | 27.342 | 20.4502 | 21.600 | 2.600 | 61.000 |
| | 11 | hours | 11 | 18.888 | 16.1917 | 13.360 | 1.900 | 53.300 |
| | 12 | hours | 10 | 14.660 | 10.6954 | 13.150 | 2.900 | 34.900 |
| | 14 | hours | 6 | 10.390 | 12.1732 | 5.800 | 2.700 | 34.540 |
| | 16 | hours | 6 | 10.223 | 10.3425 | 7.900 | 2.700 | 30.500 |
| | 20 | hours | 6 | 11.708 | 9.3859 | 9.200 | 3.500 | 28.300 |
| | 24 | hours | 7 | 15.880 | 9.7151 | 11.900 | 6.010 | 34.750 |
| Week 8 (Infusion 8) | Time ‘0’ (pre-infusion) | | 3 | 10.967 | 5.5194 | 11.500 | 5.200 | 16.200 |
| | 4 | hours into infusion | 3 | 28.700 | 12.4193 | 29.500 | 15.900 | 40.700 |
| | 8 | hours (before the end) | 4 | 27.930 | 14.7909 | 32.140 | 7.340 | 40.100 |
| | 8.25 | hours | 0 | | | | | |
| | 8.5 | hours | 3 | 11.267 | 2.9006 | 11.200 | 8.400 | 14.200 |
| | 9 | hours | 3 | 20.167 | 24.2232 | 10.100 | 2.600 | 47.800 |
| | 10 | hours | 4 | 5.803 | 5.5523 | 3.655 | 1.900 | 14.000 |
| | 11 | hours | 4 | 15.700 | 11.7266 | 14.350 | 3.900 | 30.200 |
| | 12 | hours | 4 | 14.150 | 11.6257 | 10.600 | 4.400 | 31.000 |
| | 14 | hours | 5 | 19.620 | 21.4957 | 9.900 | 2.800 | 52.900 |
| | 16 | hours | 5 | 11.680 | 8.2950 | 9.000 | 2.600 | 24.600 |
| | 20 | hours | 5 | 14.520 | 10.7369 | 15.500 | 2.200 | 30.900 |
| | 24 | hours | 5 | 16.200 | 11.5739 | 9.900 | 6.000 | 32.400 |

ATP Therapeutics, Inc.

Protocol # DMS D0005: A Phase I Study of ATP in Advanced Cancer

TABLE 14.2.11
Summary of ATP Pharmacokinetics
ITT Sample

| Visit | Time Point | | N | Mean | Std. Dev. | Median | Minimum | Maximum |
|---|---|---|---|---|---|---|---|---|
| Initial Extracellular | | | | | | | | |
| ATP Concentration | | | | | | | | |
| (microM) | | | | | | | | |
| Week 1 (Infusion 1) | Time ‘0’ (pre-infusion) | | 13 | 0.084 | 0.0996 | 0.043 | 0.005 | 0.360 |
| | 4 | hours into infusion | 13 | 0.156 | 0.1568 | 0.109 | 0.023 | 0.540 |
| | 8 | hours (before the end) | 13 | 0.491 | 0.7178 | 0.270 | 0.030 | 2.750 |
| | 8.25 | hours | 1 | 0.141 | 0.0000 | 0.141 | 0.141 | 0.141 |
| | 8.5 | hours | 10 | 0.656 | 1.0322 | 0.238 | 0.049 | 3.328 |
| | 9 | hours | 10 | 0.356 | 0.4142 | 0.150 | 0.010 | 1.170 |
| | 10 | hours | 12 | 0.269 | 0.5040 | 0.091 | 0.040 | 1.800 |
| | 11 | hours | 12 | 0.117 | 0.1389 | 0.062 | 0.010 | 0.400 |
| | 12 | hours | 11 | 0.085 | 0.0933 | 0.044 | 0.020 | 0.290 |
| | 14 | hours | 8 | 0.082 | 0.0835 | 0.029 | 0.020 | 0.230 |
| | 16 | hours | 10 | 0.080 | 0.0908 | 0.050 | 0.005 | 0.297 |
| | 20 | hours | 8 | 0.090 | 0.0877 | 0.052 | 0.008 | 0.220 |
| | 24 | hours | 12 | 0.136 | 0.1964 | 0.079 | 0.006 | 0.730 |
| Week 3 (Infusion 3) | Time ‘0’ (pre-infusion) | | 10 | 0.063 | 0.0441 | 0.055 | 0.006 | 0.160 |
| | 4 | hours into infusion | 9 | 0.786 | 1.5133 | 0.200 | 0.006 | 4.680 |
| | 8 | hours (before the end) | 9 | 0.445 | 0.5008 | 0.240 | 0.033 | 1.480 |
| | 8.25 | hours | 1 | 0.055 | 0.0000 | 0.055 | 0.055 | 0.055 |
| | 8.5 | hours | 9 | 0.511 | 0.6609 | 0.200 | 0.055 | 1.753 |
| | 9 | hours | 9 | 0.417 | 0.4708 | 0.170 | 0.040 | 1.300 |
| | 10 | hours | 9 | 0.285 | 0.4293 | 0.060 | 0.017 | 1.215 |
| | 11 | hours | 10 | 0.124 | 0.1467 | 0.060 | 0.015 | 0.449 |
| | 12 | hours | 8 | 0.088 | 0.0877 | 0.065 | 0.005 | 0.270 |
| | 14 | hours | 6 | 0.037 | 0.0218 | 0.032 | 0.013 | 0.074 |
| | 16 | hours | 5 | 0.074 | 0.0957 | 0.040 | 0.018 | 0.244 |
| | 20 | hours | 5 | 0.118 | 0.1064 | 0.070 | 0.020 | 0.243 |
| | 24 | hours | 8 | 0.058 | 0.0445 | 0.044 | 0.020 | 0.150 |
| Week 8 (infusion 8) | Time ‘0’ (pre-infusion) | | 3 | 0.350 | 0.4939 | 0.080 | 0.050 | 0.920 |
| | 4 | hours into infusion | 3 | 0.377 | 0.3102 | 0.390 | 0.060 | 0.680 |
| | 8 | hours (before the end) | 4 | 0.373 | 0.2727 | 0.320 | 0.100 | 0.750 |
| | 8.25 | hours | 0 | | | | | |
| | 8.5 | hours | 3 | 0.151 | 0.1654 | 0.080 | 0.033 | 0.340 |
| | 9 | hours | 3 | 0.274 | 0.3554 | 0.120 | 0.021 | 0.680 |
| | 10 | hours | 4 | 0.047 | 0.0387 | 0.043 | 0.013 | 0.090 |
| | 11 | hours | 4 | 0.034 | 0.0325 | 0.025 | 0.005 | 0.080 |
| | 12 | hours | 4 | 0.044 | 0.0203 | 0.040 | 0.026 | 0.070 |
| | 14 | hours | 5 | 0.080 | 0.0589 | 0.060 | 0.019 | 0.172 |
| | 16 | hours | 5 | 0.070 | 0.0581 | 0.037 | 0.020 | 0.150 |
| | 20 | hours | 5 | 0.117 | 0.1421 | 0.054 | 0.040 | 0.370 |
| | 24 | hours | 4 | 0.084 | 0.1440 | 0.016 | 0.005 | 0.300 |

ATP Therapeutics, Inc.

Protocol #DMS D0005: A Phase I Study of ATP in Advanced Cancer

TABLE 14.2.6.1
Summary of Cachexia Markers - Part I
Observed and Change from Baseline Values (1) (ITT Sample)

| | Observed Values | | | | | | Change from Baseline | | | | | |
|---|---|---|---|---|---|---|---|---|---|---|---|---|
| Visit | N | Mean | Std. Dev. | Median | Minimum | Maximum | N | Mean | Std. Dev. | Median | Minimum | Maximum |
| Serum Albumin (g/dL) | | | | | | | | | | | | |
| Week 1 (Infusion 1) | 15 | 3.3 | 0.58 | 3.4 | 2.4 | 4.2 | 15 | 0.0 | 0.00 | 0.0 | 0.0 | 0.0 |
| Week 2 (Infusion 2) | 14 | 3.2 | 0.46 | 3.2 | 2.4 | 4.0 | 14 | −0.1 | 0.28 | 0.0 | −0.7 | 0.2 |
| Week 4 (Infusion 4) | 11 | 3.2 | 0.57 | 3.0 | 2.2 | 3.9 | 11 | −0.2 | 0.31 | −0.2 | −0.6 | 0.3 |
| Week 6 (Infusion 6) | 9 | 3.2 | 0.60 | 3.1 | 1.9 | 3.9 | 9 | −0.1 | 0.42 | −0.2 | −0.5 | 0.8 |
| Week 8 (Infusion 8) | 7 | 3.3 | 0.58 | 3.4 | 2.3 | 4.0 | 7 | −0.1 | 0.72 | −0.1 | −1.1 | 1.0 |
| Week 10 (Follow-up | 6 | 3.6 | 0.36 | 3.7 | 3.0 | 3.9 | 6 | 0.0 | 0.28 | −0.1 | −0.3 | 0.4 |
| Week 13 (Follow-up) | 6 | 3.5 | 0.59 | 3.5 | 2.9 | 4.2 | 6 | −0.0 | 0.31 | −0.1 | −0.5 | 0.3 |
| Pre-albumin (mg/dL) | | | | | | | | | | | | |
| Week 1 (Infusion 1) | 15 | 19.7 | 11.92 | 18.0 | 7.0 | 55.0 | 15 | 0.0 | 0.00 | 0.0 | 0.0 | 0.0 |
| Week 2 (Infusion 2) | 13 | 19.4 | 11.21 | 17.0 | 7.0 | 44.0 | 13 | −1.2 | 5.21 | 0.0 | −11.0 | 10.0 |
| Week 4 (Infusion 4) | 10 | 16.5 | 7.47 | 17.0 | 7.0 | 29.0 | 10 | −1.3 | 4.06 | 0.0 | −11.0 | 3.0 |
| Week 6 (Infusion 6) | 8 | 16.6 | 6.78 | 16.0 | 10.0 | 29.0 | 8 | −7.5 | 11.72 | −4.5 | −34.0 | 3.0 |
| Week 8 (Infusion 8) | 7 | 19.6 | 8.40 | 19.0 | 7.0 | 31.0 | 7 | −6.3 | 17.41 | −4.0 | −37.0 | 21.0 |
| Week 10 (Follow-up) | 6 | 20.2 | 7.36 | 18.5 | 11.0 | 31.0 | 6 | −8.3 | 16.55 | −1.0 | −39.0 | 4.0 |
| Week 13 (Follow-up) | 5 | 22.4 | 8.35 | 24.0 | 13.0 | 33.0 | 5 | −8.4 | 19.24 | 0.0 | −40.0 | 7.0 |
| C-Reactive | | | | | | | | | | | | |
| Protein (mg/dL) | | | | | | | | | | | | |
| Week 1 (Infusion 1) | 15 | 5.1 | 4.78 | 3.4 | 0.3 | 14.2 | 15 | 0.0 | 0.00 | 0.0 | 0.0 | 0.0 |
| Week 2 (Infusion 2) | 14 | 5.0 | 5.11 | 3.3 | 0.4 | 14.6 | 14 | 0.1 | 2.15 | 0.5 | −5.4 | 3.6 |
| Week 4 (Infusion 4) | 9 | 6.6 | 7.62 | 6.1 | 0.4 | 24.3 | 9 | 2.5 | 5.05 | 1.6 | −5.1 | 13.3 |
| Week 6 (Infusion 6) | 8 | 4.5 | 6.32 | 2.5 | 0.4 | 19.1 | 8 | 0.2 | 7.78 | 0.0 | −10.0 | 15.7 |
| Week 8 (Infusion 8) | 6 | 9.0 | 11.07 | 4.7 | 0.4 | 27.6 | 6 | 3.7 | 12.86 | 0.0 | −9.9 | 24.2 |
| Week 10 (Follow-up) | 4 | 11.2 | 14.04 | 7.0 | 0.4 | 30.5 | 4 | 6.0 | 14.07 | −0.7 | −1.6 | 27.1 |
| Week 13 (Follow-up) | 5 | 9.1 | 15.27 | 0.4 | 0.4 | 35.7 | 5 | 5.3 | 15.29 | 0.0 | −5.8 | 32.3 |

Note:
C-Reactive Protein value <0.4 was treated as 0.4.
Pre-albumin value <7 was treated as 7.

ATP Therapeutics, Inc.

Protocol #DMS D0005: A Phase I Study of ATP in Advanced Cancer

TABLE 14.3.5.2
Summary of Laboratory Parameters: Chemistry
Observed and Change from Baseline Values (1)

| | Observed Values | | | | | | Change from Baseline | | | | | |
|---|---|---|---|---|---|---|---|---|---|---|---|---|
| | | | Std. | | | | | | Std. | | | |
| Visit | N | Mean | Dev. | Median | Minimum | Maximum | N | Mean | Dev. | Median | Minimum | Maximum |
| Phosphorus (mg/dL) | | | | | | | | | | | | |
| Week 1 (Infusion 1) | 15 | 3.1 | 0.61 | 3.2 | 1.9 | 4.3 | 15 | 0.0 | 0.00 | 0.0 | 0.0 | 0.0 |
| Week 2 (Infusion 2) | 14 | 3.2 | 0.70 | 3.2 | 1.7 | 4.3 | 14 | 0.1 | 0.51 | 0.1 | −0.9 | 1.0 |
| Week 4 (Infusion 4) | 11 | 3.1 | 0.73 | 3.0 | 2.1 | 4.6 | 11 | −0.1 | 0.52 | 0.0 | −1.1 | 0.4 |
| Week 6 (Infusion 6) | 9 | 3.1 | 0.54 | 3.3 | 2.1 | 3.7 | 9 | −0.1 | 0.73 | 0.2 | −1.1 | 0.9 |
| Week 8 (Infusion 8) | 7 | 3.4 | 0.45 | 3.4 | 2.7 | 4.0 | 7 | 0.3 | 0.51 | 0.0 | −0.3 | 0.9 |
| Week 10 (Follow-up | 6 | 3.2 | 0.57 | 3.3 | 2.6 | 3.8 | 6 | 0.0 | 0.69 | 0.3 | −1.1 | 0.7 |
| Week 13 (Follow-up) | 5 | 3.2 | 0.58 | 3.2 | 2.4 | 3.9 | 5 | −0.0 | 0.61 | 0.2 | −0.7 | 0.5 |
| LDH (U/L) | | | | | | | | | | | | |
| Week 1 (Infusion 1) | 15 | 349.0 | 210.0 | 265.0 | 137.0 | 917.0 | 15 | 0.0 | 0.00 | 0.0 | 0.0 | 0.0 |
| Week 2 (Infusion 2) | 14 | 386.6 | 221.2 | 325.5 | 133.0 | 789.0 | 14 | 53.4 | 164.2 | 24.0 | −196 | 503.0 |
| Week 4 (Infusion 4) | 11 | 277.4 | 152.1 | 211.0 | 122.0 | 547.0 | 11 | 23.4 | 100.5 | 3.0 | −97.0 | 261.0 |
| Week 6 (Infusion 6) | 9 | 265.6 | 188.7 | 173.0 | 119.0 | 639.0 | 9 | 12.2 | 106.9 | −20.0 | −111 | 217.0 |
| Week 8 (Infusion 8) | 6 | 245.0 | 201.8 | 161.0 | 115.0 | 646.0 | 6 | 24.0 | 169.6 | −25.0 | −113 | 360.0 |
| Week 10 (Follow-up) | 5 | 214.6 | 143.3 | 155.0 | 122.0 | 468.0 | 5 | 1.0 | 107.4 | −15.0 | −98.0 | 182.0 |
| Week 13 (Follow-up) | 5 | 168.2 | 43.46 | 158.0 | 125.0 | 238.0 | 5 | −41.2 | 45.34 | −27.0 | −110 | 4.0 |
| Total Bilirubin (mg/dL) | | | | | | | | | | | | |
| Week 1 (Infusion 1) | 15 | 0.7 | 0.83 | 0.4 | 0.2 | 3.2 | 15 | 0.0 | 0.00 | 0.0 | 0.0 | 0.0 |
| Week 2 (Infusion 2) | 14 | 0.6 | 0.81 | 0.4 | 0.1 | 3.3 | 14 | −0.1 | 0.29 | 0.0 | −1.0 | 0.2 |
| Week 4 (Infusion 4) | 11 | 0.8 | 1.64 | 0.2 | 0.2 | 5.7 | 11 | 0.2 | 0.79 | −0.1 | −0.3 | 2.5 |
| Week 6 (Infusion 6) | 9 | 1.9 | 4.65 | 0.3 | 0.2 | 14.3 | 9 | 1.2 | 3.71 | −0.1 | −0.3 | 11.1 |
| Week 8 (Infusion 8) | 7 | 0.4 | 0.19 | 0.4 | 0.2 | 0.7 | 7 | −0.0 | 0.20 | −0.1 | −0.2 | 0.4 |
| Week 10 (Follow-up) | 6 | 0.5 | 0.33 | 0.4 | 0.2 | 1.0 | 6 | 0.1 | 0.32 | −0.1 | −0.1 | 0.7 |
| Week 13 (Follow-up) | 6 | 0.4 | 0.19 | 0.4 | 0.2 | 0.7 | 6 | 0.1 | 0.19 | 0.1 | −0.1 | 0.4 |

ATP Therapeutics, Inc.

Protocol #DMS D0005: A Phase I Study of ATP in Advanced Cancer

TABLE 14.2.10
Summary of Skeletal Strength
Observed and Change from Baseline Values (1) (1) (ITT Sample)

| | Observed Values | | | | | | Change from Baseline | | | | | |
|---|---|---|---|---|---|---|---|---|---|---|---|---|
| | | | Std. | | | | | | Std. | | | |
| Visit | N | Mean | Dev. | Median | Minimum | Maximum | N | Mean | Dev. | Median | Minimum | Maximum |
| Skeletal Muscle | | | | | | | | | | | | |
| Strength (kg) | | | | | | | | | | | | |
| Week <1 (Screening) | 15 | 35.1 | 12.05 | 35.0 | 20.0 | 60.0 | 15 | 0.0 | 0.00 | 0.0 | 0.0 | 0.0 |
| Week 1 (Infusion 1) | 6 | 38.5 | 10.19 | 35.0 | 29.0 | 58.0 | 6 | −1.7 | 5.16 | −1.5 | −11.0 | 4.0 |
| Week 2 (Infusion 2) | 14 | 34.6 | 11.49 | 34.0 | 18.0 | 56.0 | 14 | −1.4 | 3.82 | −2.0 | −9.0 | 6.0 |
| Week 4 (Infusion 4) | 11 | 31.5 | 9.77 | 32.0 | 18.0 | 48.0 | 11 | −4.1 | 6.09 | −2.0 | −15.0 | 4.0 |
| Week 8 (Infusion 8) | 7 | 30.0 | 11.83 | 26.0 | 18.0 | 50.0 | 7 | −2.4 | 5.41 | −2.0 | −13.0 | 4.0 |
| Week 10 (follow-up | 6 | 33.7 | 21.58 | 30.0 | 12.0 | 75.0 | 6 | 1.8 | 19.68 | −3.0 | −16.0 | 40.0 |
| Week 13 (follow-up) | 4 | 32.5 | 19.00 | 26.0 | 18.0 | 60.0 | 4 | 6.3 | 17.29 | −1.0 | −5.0 | 32.0 |

Procedure for measuring voluntary (skeletal) muscle strength-hand grip strength
1. This measurement will be performed in the standing position (without support) using the patients' dominant hand in the horizontal outstretched position of 90 degrees to the body.
2. Measurements will be taken using a calibrated and well maintained hand-held commercially available dynamometer.
3. All measurements that are performed during the treatment period will be taken prior to placement of intravenous cannula and prior to study treatment administration
4. Patients will undertake the hand-grip strength maneuver after initial training—with encouragement from the observer to “do their best”. This will be performed ×3 with no more than 30 seconds between attempts.
5. The maximal value of the three attempts will be recorded as the hand-grip strength on that day.
6. The testing will be performed and documented by the same individual member of the research team (as often as is practically possible).
The dynamometer instrument will be to the greatest extent possible the same instrument for any one patient throughout the study and will be initially calibrated and checked for accuracy prior to the study and thereafter on a regular basis.
   ATP Therapeutics, Inc.

Protocol #DMS D0005: A Phase I Study of ATP in Advanced Cancer

TABLE 14.2.6.2
Summary of Cachexia Markers - Part II
Observed and Change from Baseline Values (1) (ITT Sample)

| | Observed Values | | | | | | Change from Baseline | | | | | |
|---|---|---|---|---|---|---|---|---|---|---|---|---|
| | | | Std. | | | | | | Std. | | | |
| Visit | N | Mean | Dev. | Median | Minimum | Maximum | N | Mean | Dev. | Median | Minimum | Maximum |
| Tumor necrosis | | | | | | | | | | | | |
| factor-alfa (mmol/L) | | | | | | | | | | | | |
| Week 1 (Infusion 1) | 13 | 1.8 | 1.16 | 1.7 | 0.8 | 5.4 | 13 | 0.0 | 0.00 | 0.0 | 0.0 | 0.0 |
| Week 2 (Infusion 2) | 13 | 1.9 | 0.87 | 1.8 | 0.8 | 3.7 | 12 | 0.0 | 0.67 | 0.0 | −1.8 | 1.2 |
| Week 4 (Infusion 4) | 11 | 2.0 | 0.76 | 1.8 | 0.9 | 3.2 | 10 | 0.1 | 0.94 | 0.2 | −2.2 | 1.4 |
| Week 6 (Infusion 6) | 9 | 1.7 | 0.50 | 1.8 | 1.0 | 2.5 | 8 | −0.2 | 1.13 | 0.1 | −2.9 | 0.7 |
| Week 8 (Infusion 8) | 7 | 1.6 | 0.45 | 1.5 | 1.2 | 2.6 | 6 | 0.0 | 0.35 | −0.1 | −0.2 | 0.7 |
| Week 10 (Follow-up | 5 | 1.8 | 0.49 | 1.8 | 1.3 | 2.6 | 4 | 0.2 | 0.30 | 0.2 | −0.1 | 0.6 |
| Week 13 (Follow-up) | 4 | 1.4 | 0.08 | 1.4 | 1.3 | 1.5 | 4 | 0.1 | 0.47 | −0.1 | −0.3 | 0.8 |
| Interleukin-6 (mmol/L) | | | | | | | | | | | | |
| Week 1 (Infusion 1) | 11 | 13.8 | 16.04 | 8.2 | 0.0 | 52.7 | 11 | 0.0 | 0.00 | 0.0 | 0.0 | 0.0 |
| Week 2 (Infusion 2) | 12 | 31.5 | 50.15 | 16.2 | 0.9 | 183.9 | 10 | 21.6 | 40.00 | 8.3 | −1.5 | 131.2 |
| Week 4 (Infusion 4) | 9 | 48.5 | 75.30 | 25.5 | 0.0 | 238.6 | 8 | 35.0 | 64.75 | 10.8 | −11.4 | 185.9 |
| Week 6 (Infusion 6) | 7 | 12.5 | 10.58 | 9.9 | 0.0 | 31.1 | 6 | −5.1 | 22.45 | −3.1 | −45.1 | 17.3 |
| Week 8 (Infusion 8) | 5 | 29.7 | 23.51 | 24.6 | 9.9 | 69.9 | 4 | 11.7 | 38.88 | 13.4 | −36.0 | 56.1 |
| Week 10 (Follow-up | 4 | 37.5 | 37.65 | 24.5 | 9.3 | 91.9 | 3 | 30.8 | 45.85 | 7.0 | 1.7 | 83.7 |
| Week 13 (Follow-up) | 2 | 28.1 | 33.42 | 28.1 | 4.5 | 51.8 | 2 | 22.9 | 37.62 | 22.9 | −3.7 | 49.5 |

ATP Therapeutics, Inc.

Protocol #DMS D0005: A Phase I Study of ATP in Advanced Cancer

TABLE 14.2.7
Summary of Karnofsky Performance Status
Observed and Change from Baseline Values (1) (ITT Sample)

| | Observed Values | | | | | | Change from Baseline | | | | | |
|---|---|---|---|---|---|---|---|---|---|---|---|---|
| | | | Std. | | | | | | Std. | | | |
| Visit | N | Mean | Dev. | Median | Minimum | Maximum | N | Mean | Dev. | Median | Minimum | Maximum |
| Karnofsky Performance | | | | | | | | | | | | |
| Status (%) | | | | | | | | | | | | |
| Week 1 (Infusion 1) | 15 | 78.0 | 9.41 | 80.00 | 60.0 | 90.0 | 15 | 0.0 | 0.00 | 0.0 | 0.0 | 0.0 |
| Week 2 (Infusion 2) | 14 | 77.1 | 8.25 | 75.0 | 70.0 | 90.0 | 14 | −2.1 | 5.79 | 0.0 | −20.0 | 0.0 |
| Week 3 (Infusion 3) | 13 | 76.9 | 10.32 | 80.0 | 60.0 | 90.0 | 13 | −2.3 | 4.39 | 0.0 | −10.0 | 0.0 |
| Week 4 (Infusion 4) | 11 | 78.2 | 9.82 | 80.0 | 60.0 | 90.0 | 11 | −0.9 | 3.02 | 0.0 | −10.0 | 0.0 |
| Week 5 (Infusion 5) | 10 | 71.0 | 16.63 | 75.0 | 30.0 | 90.0 | 10 | −7.0 | 12.52 | 0.0 | −40.0 | 0.0 |
| Week 6 (Infusion 6) | 9 | 74.4 | 7.26 | 80.0 | 60.0 | 80.0 | 9 | −4.4 | 5.27 | 0.0 | −10.0 | 0.0 |
| Week 7 (Infusion 7) | 7 | 72.9 | 7.56 | 70.0 | 60.0 | 80.0 | 7 | −4.3 | 7.87 | 0.0 | −20.0 | 0.0 |
| Week 8 (Infusion 8) | 7 | 71.4 | 9.00 | 70.0 | 60.0 | 80.0 | 7 | −5.7 | 5.35 | −10.0 | −10.0 | 0.0 |
| Week 10 (Follow-up | 6 | 73.3 | 10.33 | 70.0 | 60.0 | 90.0 | 6 | −5.0 | 10.49 | −5.0 | −20.0 | 10.0 |
| Week 13 (Follow-up) | 5 | 76.0 | 11.40 | 80.0 | 60.0 | 90.0 | 5 | −4.0 | 11.40 | 0.0 | −20.0 | 10.0 |

KARNOFSKY ACTIVITY SCALE

| FUNCTIONAL STATUS | RATING | GROUP SCORES |
|---|---|---|
| Normal, no complaints; no evidence of | 100 | Rehabilitated |
| disease able to carry on normal activity; | 90 | (80+) |
| minor signs of symptoms of disease | | |
| Normal activity with effort: some signs | 80 | |
| of symptoms of disease | | |
| Cares for self; unable to carry on | 70 | Self-care only |
| normal activity or do active work | | (70-79) |
| Requires occasional assistance but able | 60 | Requires caretaker |
| to care for most needs | | (40-69) |
| Requires considerable assistance and | 50 | |
| frequent medical care | | |
| Disabled; requires special care and | 40 | |
| assistance | | |
| Severely disabled; hospitalization is | 30 | Requires |
| indicated although death not imminent | 20 | Institutionalization |
| Very sick; hospitalization necessary | 10 | (1-39) |
| Moribund; fatal processes progressing | | |
| Dead | 0 | |

From: Yates JW, Chalmber B, McKegney FP. Evaluation of patients with advanced cancer using the Karnofsky Performance Status. Cancer 45: 2220-2224 (1980).

According to the above disclosure and teaching, variations of the present invention are possible. It is therefore understood that the present invention may be practiced in ways other than specifically described. Having thus described my invention, what I claim as useful and desire to secure by Letters Patent is:

1. A method for treating an aging individual and/or patient suffering from advanced diseases wherein said individuals and patients exhibit negative prognostic factors for survival and quality of life by administering at least one agent selected from a group consisting of adenosine, adenosine 5′-monophosphate, adenosine 5′-diphosphate, adenosine 5′-triphosphate, pharmaceutically acceptable salts thereof, liposomes thereof, metal cation complexes thereof chelates thereof, and radionuclide complexes thereof.

2. The method according to claim 1, wherein levels of negative prognostic factors for survival and quality of life in need of treatment are selected from the group consisting of low serum albumin, low serum bilirubin, high serum lactate dehydrogenase (LDH), high blood tumor necrosis factor-alpha (TNF-alpha), low skeletal muscle strength and low Karnofsky performance status.

3. A method according to claim 1 which comprises treating said individual and/or patient for at least one condition selected from the group consisting of inflammatory bowel diseases, chronic heart diseases, chronic obstructive pulmonary disease, sepsis, acute lung injury, rheumatoid arthritis, osteoarthritis, advanced refractory cancer, severe trauma and injury.

4. The method according to claim 1 wherein adenosine and/or adenosine 5′-monophosphate and/or adenosine 5′-triphosphate are administered to an aging human and/or a human patient in need thereof on an out-patient basis.

5. The method according to claim 4 wherein adenosine and/or adenosine 5′-monophosphate and/or adenosine 5′-triphosphate are administered to an aging human and/or a patient in need thereof in an out-patient clinic.

6. The method according to claim 4 wherein adenosine and/or adenosine 5′-monophosphate and/or adenosine 5′-triphosphate are administered to an aging human and/or a human patient in need thereof at home using standard home care.

7. The method according to claim 1 wherein treating is with adenosine 5′-triphosphate as an active agent.

8. The method according to claim 3 wherein treating is with adenosine 5′-triphosphate as an active agent.

9. The method according to claim 8 wherein inflammatory bowel diseases are treated with adenosine 5′-triphosphate.

10. The method according to claim 8 wherein chronic heart diseases are treated with adenosine 5′-triphosphate.

11. The method according to claim 8 wherein chronic obstructive pulmonary disease is treated with adenosine 5′-triphosphate.

12. The method according to claim 8 wherein sepsis and/or acute lung injury are treated with adenosine 5′-triphosphate.

13. The method according to claim 8 wherein rheumatoid arthritis is treated with adenosine 5′-triphosphate.

14. The method according to claim 8 wherein osteoarthritis is treated with adenosine 5′-triphosphate.

15. The method according to claim 8 wherein advanced refractory cancer is treated with adenosine 5′-triphosphate.

16. The method according to claim 8 wherein severe trauma and/or injury are treated with adenosine 5′-triphosphate.

17. The method according to claim 7 wherein the amount of adenosine 5′-triphosphate is about 1-150 micrograms per kilogram of body weight per minute and administering is by infusion.

18. The method according to claim 7 wherein the amount of adenosine 5′-triphosphate is about 0.01-50 milligrams per kilogram of body weight per 24 hours and administering is by injection.

19. The method according to claim 7 wherein the amount of adenosine 5′-triphosphate is about 0.01-50 milligrams per kilogram of body weight per 24 hours and administering is oral or sublingual.

20. The method according to claim 19 wherein an oral or sublingual composition of adenosine 5′-triphosphate is administered in a pill form, a tablet form, a capsule form, a soft gel form, a lozenge form or other oral therapeutic composition containing adenosine 5′-triphosphate binders, stabilizers, fillers and enteric coating materials.

21. The method according to claim 7 wherein the amount of adenosine 5′-triphosphate is about 0.01-50 milligrams per kilograms of body weight per 24 hours and administering is topical.

22. A process of treating levels of negative prognostic factors for survival and quality of life in an aging individual and/or in a patient suffering from advanced diseases by increasing liver, blood and blood plasma pools of adenosine 5′-triphosphate in said aged individual and/or patient in need thereof.

23. The process of claim 22 wherein said aging individual and/or human patient with negative prognostic factors in need of treatment suffers from low serum albumin, low serum bilirubin, high serum lactate dehydrogenase (LDH), high blood tumor necrosis factor-alpha (TNF-alpha), low skeletal muscle strength and low Karnofsky performance status.

24. The process of claim 23 wherein treating an aging individual and/or a patient is with an effective amount of an agent selected from a group consisting of adenosine and/or adenosine 5′-monophosphate and/or adenosine 5′-diphosphate and/or adenosine 5′-triphosphate, pharmaceutically acceptable salts thereof, liposomes thereof, metal cation complexes thereof, chelates thereof, and radionuclide complexes thereof.

25. The process according to claim 23 wherein treatment for alleviating symptoms is utilized by treating an aging individual and/or patient suffering from an advanced disease and/or condition selected from the group consisting of inflammatory bowel diseases, chronic heart diseases, chronic obstructive pulmonary disease, sepsis and/or acute lung injury, rheumatoid arthritis, osteoarthritis, advanced refractory cancer and severe trauma and/or injury.

26. The process according to claim 24 wherein said aging individual and/or patient in need thereof are treated in an out-patient setting.

27. The process according to claim 24 wherein said aging individual and/or patient in need thereof are treated on a home care basis.

28. The process according to claim 24 wherein said aging individual and/or patient in need thereof is treated with adenosine 5′-triphosphate.

29. The process according to claim 28 wherein said aging individual and/or patient in need thereof is treated with a dose of about 1-150 micrograms per kilogram of body weight per minute and administering is by infusion.

30. The process according to claim 28 wherein said aging individual and/or patient in need thereof is treated with a dose of about 0.01-50 milligrams per kilogram of body weight per 24 hours and administering is by injection.

31. The process according to claim 28 wherein said aging individual and/or patient in need thereof are treated with a dose of about 0.01-50 milligrams per kilogram of body weight per 24 hours and administering is oral or sublingual.

32. The process according to claim 31 which comprises administering an oral or sublingual composition of adenosine 5′-triphosphate is administered in a pill form, a tablet form, a capsule form, a soft gel form, a lozenge form or other oral therapeutic composition containing adenosine 5′-triphosphate, binders, stabilizers, fillers and enteric coating materials.

33. The process according to claim 28 wherein said aging individual and/or patient in need thereof is treated with a dose of about 0.01-50 milligrams per kilograms of body weight per 24 hours and administering is topical.
